(19) United States
(12) Patent Application Publication
(10) Pub. No.: US 2025/0121006 A1
(43) Pub. Date: Apr. 17, 2025

(54) THERAPEUTIC COMBINATIONS COMPRISING ANTI-STEAP2 CHIMERIC ANTIGEN RECEPTOR T CELLS

(71) Applicant: AstraZeneca AB, Sodertalje (SE)

(72) Inventors: Peter ZANVIT, Gaithersburg, MD (US); Emily BOSCO, Gaithersburg, MD (US); Kelly MCGLINCHEY, Gaithersburg, MD (US); Jessica PEZOLD, Gaithersburg, MD (US)

(21) Appl. No.: 18/896,133

(22) Filed: Sep. 25, 2024

Related U.S. Application Data
(60) Provisional application No. 63/585,292, filed on Sep. 26, 2023.

(51) Int. Cl. A61K 35/17; A61K 31/4166; A61K 31/58; A61K 39/00; A61P 35/00; C07K 14/71; C07K 14/725; C07K 16/30

(52) U.S. Cl. CPC A61K 35/17; A61K 31/4166; A61K 31/58; A61K 40/11; A61K 40/31; A61K 40/4274; A61P 35/00; C07K 14/7051; C07K 14/71; C07K 16/3069; A61K 2239/13; C07K 2317/565

(57) ABSTRACT

The disclosure provides therapeutic combinations of chimeric antigen receptor T cells that specifically bind human STEAP2 (e.g., AZD0754) with androgen receptor antagonists (e.g., enzalutamide). Methods of administering the combinations to treat cancer (e.g., prostate cancer) are also provided.

### REFERENCE TO SEQUENCE LISTING SUBMITTED ELECTRONICALLY VIA EFS WEB

The content of the electronically submitted sequence listing (CARTSTEAP2-101-US-PSP_ST26; Size: 30,921 bytes; and Date of Creation: Sep. 21, 2023) submitted in this application is incorporated herein by reference in its entirety.

### BACKGROUND OF THE DISCLOSURE

Prostate cancer (PC) is the second most frequent cancer worldwide and is one of the leading causes of male cancer-related deaths. Approximately 1 in 8 men will be diagnosed with prostate cancer, and about 1 in 41 men will die of this cancer (American Cancer Society, 2023). Fortunately, PC progression is usually slow and the prognosis generally favorable when a suitable treatment is initiated early and the tumor is confined to prostate-tissue. On the other hand, for patients who are not cured after an effective primary intervention such as radical prostatectomy, androgen-deprivation therapy (ADT) may control the tumor burden for years, but frequently, castrate-resistant and eventually metastatic prostate cancer emerge. Prostate cancer claims tens of thousands of lives in the US each year.

For patients with castrate-resistant prostate cancer (CRPC) with detectable metastatic disease, standard of care treatment (chemotherapy with docetaxel and prednisone in combination) prolongs survival but is not curative (Adamo et al, Front. Endocrinol., 3:73, 2012; Saad and Hotte, Can. Urol. Assoc. J., 4(6):380-384, 2010). Underlying reasons may be related to the mechanism of action of docetaxel, a microtubule-stabilizer, which preferentially kills rapidly proliferating cells but likely spares slow cycling cancer cells. Among the latter cells may be cancer stem cells, which are proposed to give rise to the majority of the malignant cells within a tumor and appear to be particularly resistant to therapy (Kong et al., Cancers (Basel), 3(1):716-729, 201 1; Lang et al, J Pathol, 217(2):299-306, 2009).

Thus, there is a need in the art for improved treatment and prevention strategies for prostate cancer. The present invention addresses this need and others. Described herein are novel methods of using chimeric antigen receptor (CAR)-expressing T cells directed to human STEAP2, in combination with androgen receptor antagonists, in the treatment of cancer, e.g., prostate cancer.

### SUMMARY OF THE DISCLOSURE

The present disclosure is directed to a method for inhibiting the growth of a tumor cell comprising contacting said tumor cell with: a T cell comprising (i) a polynucleotide encoding a chimeric antigen receptor (CAR) that binds an epitope on human six transmembrane epithelial antigen of prostate-2 (STEAP2); and at least one androgen receptor antagonist, in an amount effective to inhibit tumor cell growth; wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

Some aspects of the present disclosure are direct to a method for treating a cancer comprising tumor cells in a subject comprising administering to said subject in need thereof a therapeutically effective amount of: a T cell comprising (i) a polynucleotide encoding a chimeric antigen receptor (CAR) that binds an epitope on human six transmembrane epithelial antigen of prostate-2 (STEAP2); and at least one androgen receptor antagonist; wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

In some aspects, the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8.

In some aspects, the VH comprises the amino acid sequence set forth in SEQ ID NO: 7, and the VL comprises the amino acid sequence set forth in SEQ ID NO: 8.

In some aspects, the polynucleotide further encodes an armoring molecule and said armoring molecule comprises a dominant-negative TGF-β receptor type 2 (TGFβRIIDN).

In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10.

In some aspects, the armoring molecule comprises the amino acid sequence set forth in SEQ ID NO: 10.

In some aspects, the polynucleotide encoding the CAR comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 11.

In some aspects, the polynucleotide encoding the CAR comprises the nucleotide sequence set forth in SEQ ID NO: 11.

In some aspects, the polynucleotide encoding the CAR comprises the nucleotide sequence set forth in SEQ ID NO: 11, and the polynucleotide encoding the armoring molecule comprises the nucleotide sequence set forth in SEQ ID NO: 9.

In some aspects, the polynucleotide encoding the CAR and the polynucleotide encoding the armoring molecule are operably linked under the control of a single promoter.

In some aspects, the polynucleotide encoding the CAR and the polynucleotide encoding the armoring molecule are operably linked by an IRES.

In some aspects, the polynucleotide encoding the CAR and the polynucleotide encoding the armoring molecule are linked by a nucleotide sequence encoding a cleavable peptide linker. In some aspects, the cleavable peptide linker is a self-cleaving peptide linker. In some aspects, the cleavable peptide linker comprises a T2A peptide. In some aspects, the cleavable peptide linker comprises SEQ ID NO: 13.

In some aspects, the polynucleotide comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 14.

In some aspects, the polynucleotide comprises the nucleotide sequence set forth in SEQ ID NO: 14.

In some aspects, the T cell comprises an amino acid sequence that comprises the amino acid sequence set forth in SEQ ID NO: 12.

In some aspects, the T cell comprises an amino acid sequence that comprises the amino acid sequence set forth in SEQ ID NO: 15.

In some aspects, the androgen receptor antagonist is enzalutamide, apalutamide, darolutamide, abiraterone, bicalutamide, nilutamide, flutamide, proxalutamide, or a combination thereof.

In some aspects, the androgen receptor antagonist is enzalutamide.

In some aspects, the androgen receptor antagonist is abiraterone.

In some aspects, the androgen receptor antagonist is administered orally.

In some aspects, the T cell is administered intravenously.

In some aspects, the T cell and the androgen receptor antagonist are administered sequentially or simultaneously, and in any order.

In some aspects, administration of the androgen receptor antagonist does not inhibit T cell viability or T cell killing capacity.

In some aspects, administration of the androgen receptor antagonist does not inhibit interferon-gamma (IFNγ) release in tumor cells.

In some aspects, administration of the androgen receptor antagonist does not reduce CAR expression on the T cell.

In some aspects, the T cell is a CD8+ T cell.

In some aspects, the tumor cells are prostate tumor cells, optionally wherein the prostate tumor cells are metastatic, recurrent, or relapsed.

In some aspects, the administration increases STEAP2 expression in the tumor cells as compared to the expression of STEAP2 in the tumor cells when the T-cell is administered alone. In some aspects, the administration increases STEAP2 expression by from about 50% to about 200% in the tumor cells as compared to the expression of STEAP2 in the tumor cells when the T-cell is administered alone. In some aspects, the STEAP2 expression is increased by about 100%.

Some aspects of the present disclosure are directed to a use of a T cell and an androgen receptor antagonist in a method for inhibiting the growth of a tumor cell, the method comprising contacting said tumor cell with the T cell and at least one androgen receptor antagonist, in an amount effective to inhibit tumor cell growth, wherein the T cell comprises (i) a polynucleotide encoding a CAR that binds an epitope on human STEAP2 and (ii) a polynucleotide encoding an armoring molecule, wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6. In some aspects, the tumor cells are prostate tumor cells. In some aspects, the prostate tumor cells are metastatic, recurrent, or relapsed.

Some aspects of the present disclosure are directed to a use of a T cell and an androgen receptor antagonist in a method of treating a cancer comprising tumor cells in a subject comprising administering to said subject in need thereof a therapeutically effective amount of the T cell and at least one androgen receptor antagonist, wherein the T cell comprises (i) a polynucleotide encoding a CAR that binds an epitope on human STEAP2 and (ii) a polynucleotide encoding an armoring molecule, wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6. In some aspects, the tumor cells are prostate tumor cells. In some aspects, the prostate tumor cells are metastatic, recurrent, or relapsed.

### BRIEF DESCRIPTION OF THE DRAWINGS/FIGURES

FIGS. 1A-1C show responses of three LuCaP prostate cancer patient-derived xenograft (PDX) models to enzalutamide treatment over time. Tumor volume was measured for up to 42 days following treatment with either a vehicle control or enzalutamide (10, 30, 50, or 80 mg/kg doses) in LuCaP73 (FIG. 1A), LuCaP70 (FIG. 1B), and LuCaP86.2 (FIG. 1C) PDX models.

FIGS. 2A-2B. FIG. 2A shows the response of a LuCaP73 PDX model to varying doses of enzalutamide treatment over time. Tumor volume was measured for up to 42 days following treatment with either a vehicle control or enzalutamide (10, 30, 50, or 80 mg/kg doses). *** indicates p<0.001 and **** indicates p<0.0001 (two-sided, unadjusted p-values). FIG. 2B shows the body weight of a LuCaP73 PDX model following treatment with either a vehicle control or enzalutamide (10, 30, 50, or 80 mg/kg doses).

FIGS. 3A-3B show LuCaP73 tumor expression of androgen receptor (AR; FIG. 3A) and STEAP2 (FIG. 3B) at day 42 following treatment with vehicle or 10 mg/kg enzalutamide (Enza). AR and STEAP2 expression levels were normalized to GAPDH levels.

FIGS. 4A-4B. FIG. 4A shows the response of a LuCaP70 PDX model to varying doses of enzalutamide treatment over time. Tumor volume was measured for 21 days following treatment with either a vehicle control or enzalutamide (10, 30, 50, or 80 mg/kg doses). *** indicates p<0.001 and * indicates p<0.05 (two-sided, unadjusted p-values). FIG. 4B shows the body weight of a LuCaP 70 PDX model following treatment with either a vehicle control or enzalutamide (10, 30, 50, or 80 mg/kg doses).

FIGS. 5A-5E. FIGS. 5A-5C show LuCaP70 tumor expression of androgen receptor (AR; FIG. 5A), STEAP2 (FIG. 5B), and KLK3 (FIG. 5C) at day 21 following treatment with vehicle or enzalutamide (10, 30, 50, or 80 mg/kg doses). **** indicates p<0.0001; ** indicates p<0.01; and ns indicates not significant (determined via one-way ANOVA followed by Dunnett's multiple comparisons test). FIGS. 5D-5E show STEAP2 expression levels in LuCaP70 tumors based on immunohistochemistry (IHC) using an anti-STEAP2 antibody and calculated intensity of tumor membrane staining and percentage of cells stained (H-score; FIG. 5D) and by percent membrane staining in tumors (FIG. 5E). * indicates p<0.05 (determined via one-way ANOVA followed by Dunnett's multiple comparisons test).

FIGS. 6A-6E. FIG. 6A shows the response of a LuCaP86.2 PDX model to varying doses of enzalutamide treatment over time. Tumor volume was measured for 25 days following treatment with either a vehicle control or enzalutamide (10, 30, 50, or 80 mg/kg doses). ** indicates P<0.01, and * indicates p<0.05 (two-sided, unadjusted p-values). FIG. 6B shows the body weight of a LuCaP86.2 PDX model following treatment with either a vehicle control or enzalutamide (10, 30, 50, or 80 mg/kg doses). FIGS. 6C-6E show LuCaP86.2 tumor expression of androgen receptor (AR; FIG. 6C), STEAP2 (FIG. 6D), and KLK3 (FIG. 6E) at day 25 following treatment with vehicle or enzalutamide (10, 30, 50, or 80 mg/kg doses).

FIGS. 7A-7D show the response of enzalutamide treatment on serum levels of prostate-specific antigen (PSA) in castration-sensitive LuCaP PDX models. LuCaP73 (FIG. 7A), LuCaP70 (FIG. 7B), and LuCaP86.2 (FIG. 7C) PDX models were treated with either vehicle or enzalutamide (10, 30, 50, or 80 mg/kg doses) and serum PSA levels were determined by ELISA at the end of the study (LuCaP73=Day 42, LuCaP70=Day 21, and LuCaP86.2=Day 25). **** indicates p<0.0001 and * indicates p<0.05 (determined via one-way ANOVA followed by Dunnett's multiple comparisons test). FIG. 7D shows the effect on STEAP2 antigen binding capacity (ABC) after continuous enzalutamide treatment in an LNCAP cell line through day 21 and the effects through day 28.

FIGS. 8A-8B show the response of AZD0754 CAR T cell efficacy in a LuCaP73 PDX model. Tumor volume (FIG. 8A) and body weight (FIG. 8B) was measured over time for mice treated with untransduced T cell (UT; 5×10⁶ cells/mouse), 40A3 dnTFGβRII (i.e., AZD0754) CAR T cells (CAR T; 5×10⁵ cells/mouse), or 40A3 dnTFGβRII CAR T cells (CAR T; 5×10⁶ cells/mouse). UT/CAR T treatment was administered at the indicated point in time (day 34). N=12 for each treatment group. **** indicates p<0.0001 and * indicates p<0.05.

FIGS. 9A-9B show the response of AZD0754 CAR T cell and enzalutamide combination therapy in a LuCaP73 PDX model. Tumor volume (FIG. 9A) and body weight (FIG. 9B) was measured over time for untreated mice, mice with vehicle control, mice treated with untransduced T cell (UT; 5×10⁵ cells/mouse), mice treated with untransduced T cell (UT; 5×10⁵ cells/mouse) and vehicle control, mice treated with enzalutamide (10 mg/kg), mice treated with untransduced T cells (UT; 5×10⁵ cells/mouse) and enzalutamide (10 mg/kg), mice treated with AZD0754 (5×10⁵ cells/mouse), mice treated with AZD0754 (5×10⁵ cells/mouse) and vehicle control, and mice treated with AZD0754 (5×10⁵ cells/mouse) and enzalutamide (10 mg/kg). N=9 for each treatment group. * indicates p<0.05.

FIGS. 10A-10B show the percentage (FIG. 10A) and cell number (FIG. 10B) of AZD0754 CAR-T cells at day 42 in the blood of untreated mice, mice with vehicle control, mice treated with untransduced T cell (UT; 5×10⁵ cells/mouse), mice treated with untransduced T cell (UT; 5×10⁵ cells/mouse) and vehicle control, mice treated with enzalutamide (10 mg/kg), mice treated with untransduced T cells (UT; 5×10⁵ cells/mouse) and enzalutamide (10 mg/kg), mice treated with AZD0754 (5×10⁵ cells/mouse), mice treated with AZD0754 (5×10⁵ cells/mouse) and vehicle control, and mice treated with AZD0754 (5×10⁵ cells/mouse) and enzalutamide (10 mg/kg).

FIGS. 11A-11B show the percentage (FIG. 11A) and cell number (FIG. 11B) of hCD45+ Paratope+ TGFβRII+ CAR-T cells at day 42 in the blood of untreated mice, mice with vehicle control, mice treated with untransduced T cell (UT; 5×10⁵ cells/mouse), mice treated with untransduced T cell (UT; 5×10⁵ cells/mouse) and vehicle control, mice treated with enzalutamide (10 mg/kg), mice treated with untransduced T cells (UT; 5×10⁵ cells/mouse) and enzalutamide (10 mg/kg), mice treated with AZD0754 (5×10⁵ cells/mouse), mice treated with AZD0754 (5×10⁵ cells/mouse) and vehicle control, and mice treated with AZD0754 (5×10⁵ cells/mouse) and enzalutamide (10 mg/kg).

FIG. 12 shows the levels of serum PSA at day 42 in untreated mice, mice with vehicle control, mice treated with untransduced T cell (UT; 5×10⁵ cells/mouse), mice treated with untransduced T cell (UT; 5×10⁵ cells/mouse) and vehicle control, mice treated with enzalutamide (10 mg/kg), mice treated with untransduced T cells (UT; 5×10⁵ cells/mouse) and enzalutamide (10 mg/kg), mice treated with AZD0754 (5×10⁵ cells/mouse), mice treated with AZD0754 (5×10⁵ cells/mouse) and vehicle control, and mice treated with AZD0754 (5×10⁵ cells/mouse) and enzalutamide (10 mg/kg).

FIGS. 13A-13B show the effect of abiraterone (FIG. 13A) and enzalutamide (FIG. 13B) treatment on LNCAP or 22RV1 prostate cell line proliferation.

FIGS. 14A-14B show the effect of abiraterone (FIG. 14A) and enzalutamide (FIG. 14B) treatment on 40A3 dnTFGβRII CAR T cell proliferation following 24, 48, or 72 hours post-treatment.

FIGS. 15A-15B show the effect of abiraterone (FIG. 15A) and enzalutamide (FIG. 15B) treatment on STEAP2 expression measured by flow cytometry in LNCAP cells following 24, 48, and 72 hours post-treatment.

FIGS. 16A-16C show the effect of abiraterone treatment on 40A3 dnTFGβRII CAR T cell killing capacity (FIG. 16A) or IFNγ release by the CAR T cells (FIG. 16B) in co-culture with LNCAP cells. 5 uM abiraterone treatment was compared to DMSO, and CAR T cells were compared to untransduced T cells (UT), wherein the LNCAP and/or CAR T or UT cells were pre-treated (PreTx) with abiraterone. Androgen receptor inhibition in LNCAP cells treated with either media, DMSO, or 5 uM abiraterone was validated by qPCR of downstream target expression (FIG. 16C).

FIGS. 17A-17C show the effect of enzalutamide treatment on 40A3 dnTFGβRII CAR T cell killing capacity (FIG. 17A) or IFNγ release by the CAR T cells (FIG. 17B) in co-culture with LNCAP cells. 5 uM enzalutamide treatment was compared to DMSO, and CAR T cells were compared to untransduced T cells (UT), wherein the LNCAP and/or CAR T or UT cells were pre-treated (PreTx) with enzalutamide. Androgen receptor inhibition in LNCAP cells treated with either media, DMSO, or 5 uM enzalutamide was validated by qPCR of downstream target expression (FIG. 17C).

### DETAILED DESCRIPTION OF DISCLOSURE

The present disclosure relates to antigen-binding moieties that specifically bind an epitope on human six transmembrane epithelial antigen of prostate-2 (STEAP2). Some aspects of the present disclosure are directed to polynucleotides comprising a nucleotide sequence encoding a CAR, wherein the CAR comprises an antigen-binding domain that binds an epitope on human STEAP2. Some aspects of the present disclosure are directed to a host cell comprising the polynucleotide. Other aspects of the present disclosure are directed to antibodies or antigen-binding portions thereof that specifically bind an epitope on human STEAP2. In some aspects, the antigen-binding domain binds an epitope on an extracellular loop of human STEAP2. Further aspects of the present disclosure are directed to methods of treating a subject in need thereof comprising administering the polynucleotide, the cell, and/or the antibody or antigen-binding portion thereof to the subject. In some aspects, the subject is afflicted with a prostate cancer or a tumor derived from a prostate cancer.

### I. Terms

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided. As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety.

The term “about” is used herein to mean approximately, roughly, around, or in the regions of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower).

As used herein, the term “approximately,” as applied to one or more values of interest, refers to a value that is similar to a stated reference value. In certain aspects, the term “approximately” refers to a range of values that fall within 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The term “antibody” refers, in some aspects, to a protein comprising at least two heavy (H) chains and two light (L) chains inter-connected by disulfide bonds. Each heavy chain is comprised of a heavy chain variable region (abbreviated herein as VH) and a heavy chain constant region (abbreviated herein as CH). In some antibodies, e.g., naturally-occurring IgG antibodies, the heavy chain constant region is comprised of a hinge and three domains, CH1, CH2 and CH3. In some antibodies, e.g., naturally-occurring IgG antibodies, each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain may have the C-terminal lysine or not. Unless specified otherwise herein, the amino acids in the variable regions are numbered using the Kabat numbering system and those in the constant regions are numbered using the EU system.

An immunoglobulin can be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. In some aspects, the antibodies described herein are of the IgG1 subtype. Immunoglobulins, e.g., IgG1, exist in several allotypes, which differ from each other in at most a few amino acids. “Antibody” includes, by way of example, both naturally-occurring and non-naturally-occurring antibodies; monoclonal and polyclonal antibodies; chimeric and humanized antibodies; human and nonhuman antibodies and wholly synthetic antibodies.

The term “antigen-binding portion” of an antibody, as used herein, refers to one or more fragments of an antibody that retain the ability to specifically bind to an antigen (e.g., human STEAP2). The antigen-binding function of an antibody can be performed by fragments of a full-length antibody. Examples of binding fragments encompassed within the term “antigen-binding portion” of an antibody, e.g., an anti-STEAP2 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

The term “chimeric antigen receptor” or “CAR,” as used herein, refers to an engineered antigen-binding polypeptide, comprising an antigen-binding domain, a transmembrane domain, and an intracellular signaling domain. Expression of a CAR on the surface of a cell, e.g., an immune cell, allows the cell to target and bind a particular antigen. In some aspects, the CAR is expressed by an immune cell, e.g., a T cell. In some aspects, the antigen binding domain comprises an Fab, Fab′, F(ab′)2, Fd, Fv, single-chain fragment variable (scFv), single chain antibody, VHH, vNAR, nanobody (single-domain antibody), or any combination thereof. In some aspects, the transmembrane domain comprises a transmembrane domain selected from the transmembrane domain of CD4, CD8α, or CD28. In some aspects, the intracellular domain comprises a costimulatory domain or a portion thereof. In some aspects, the intracellular domain comprises a costimulatory domain selected from the group consisting of the intracellular domain of CD3z, a CD28 co-stimulatory domain, a CD27 co-stimulatory domain, a 4-1BB co-stimulatory domain, an ICOS co-stimulatory domain, an OX-40 co-stimulatory domain, a GITR co-stimulatory domain, a CD2 co-stimulatory domain, an IL-2Rβ co-stimulatory domain, an MyD88/CD40a CD28 co-stimulatory domain, and any combination thereof. A CAR can further comprise a “hinge” or “spacer” domain. Non-limiting examples of hinge/spacer domains include immunoglobulin hinge/spacer domains, such as an IgG1 hinge domain, and IgG2 hinge domain, an IgG3 hinge domain, or an IgG4 hinge domain.

As used herein, the term “armoring” refers to molecular manipulation of a CAR-expressing cell (e.g., a CAR-T cell) to further express one or more “armoring molecules” that can counter immunosuppression. For example, investigators recently reported modifying CAR-T cells to secrete PD-1-blocking single-chain variable fragments (scFv), which improved CAR-T cell anti-tumor activity in mouse models of PD-L1+ hematologic and solid tumors (Rafiq, S., Yeku, O., Jackson, H. et al. Targeted delivery of a PD-1-blocking scFv by CAR-T cells enhances anti-tumor efficacy in vivo. Nat Biotechnol 36, 847-856 (2018)). Others studies have demonstrated the effectiveness of armoring T cells with a dominant-negative TGF-β receptor type 2 (TGFβRIIDN) armoring molecule to neutralize the suppressive effects of TGF-β on T cells (Bollard et al., Tumor-Specific T-Cells Engineered to Overcome Tumor Immune Evasion Induce Clinical Responses in Patients With Relapsed Hodgkin Lymphoma, J Clin Oncol 36(11):1128-1139 (2018)). Currently, at least one clinical study is investigating the effectiveness of armoring anti-PSMA-CAR-T cells with a TGFβRIIDN armoring molecule for treating castrate-resistant prostate cancer (NCT03089203).

The term “STEAP2,” as used herein, refers to six-transmembrane epithelial antigen of prostate 2. STEAP2 is an integral, six-transmembrane-spanning protein that is highly expressed in prostate epithelial cells and is a cell-surface marker for prostate cancer, for example STEAP2 was found to be expressed in significant levels on an LNCaP prostate cell line (Porkka, et al. Lab Invest 2002, 82:1573-1582). STEAP2 (UniProtKB/Swiss-Prot: Q8NFT2.3) is a 490-amino acid protein encoded by STEAP2 gene located at the chromosomal region 7q21 in humans.

A “drug-resistant androgen receptor” is a modified (relative to wildtype) androgen receptor that is inhibited less effectively by the drug than a wildtype androgen receptor. A “drug-resistant human androgen receptor” is a modified (relative to wildtype) human androgen receptor that is inhibited less effectively by the drug than a wildtype human androgen receptor. Examples of a “drug-resistant human androgen receptor” include a human androgen receptor with a level of activity that is less inhibited by a competitive inhibitor (e.g., Casodex, Flutamide, MDV3100, or ARN-509) then a wildtype human androgen inhibitor, a human androgen receptor that is active without binding a ligand, and a human androgen receptor that is active without a portion or all of the ligand binding domain.

The term “androgen receptor” or “AR” or “NR3C4” refers to a nuclear receptor activated by binding of the androgenic hormone testosterone or dihydrotestosterone. The term “androgen receptor” may refer to the nucleotide sequence or protein sequence of human androgen receptor (e.g., Entrez 367, Uniprot P10275, RefSeq NM_000044, or RefSeq NP_000035 (SEQ ID NO:2)). The term “androgen receptor” includes both the wild-type form of the nucleotide sequences or proteins as well as any mutants thereof. In some aspects, “androgen receptor” is wild-type androgen receptor. In some aspects, “androgen receptor” is one or more mutant forms. The term “androgen receptor” XYZ refers to a nucleotide sequence or protein of a mutant androgen receptor wherein the Y numbered amino acid of androgen receptor that normally has an X amino acid in the wildtype, instead has a Z amino acid in the mutant. In aspects, an androgen receptor is the human androgen receptor. In aspects, the androgen receptor has the nucleotide sequence corresponding to reference number GI:349501065. In aspects, the androgen receptor has the nucleotide sequence corresponding to RefSeq NM_000044.3. In aspects, the androgen receptor has the protein sequence corresponding to reference number GI:21322252. In aspects, the androgen receptor has the protein sequence corresponding to RefSeq NP_000035.2

As used herein, the term “affinity” refers to a measure of the strength of the binding of an antigen or target (such as an epitope) to its cognate binding domain (such as a paratope). As used herein, the term “avidity” refers to the overall stability of the complex between a population of epitopes and paratopes (i.e., antigens and antigen binding domains).

The term “epitope” refers to a site on an antigen (e.g., STEAP2) to which a chimeric antigen receptor, immunoglobulin, or antibody specifically binds, e.g., as defined by the specific method used to identify it. Epitopes can be formed both from contiguous amino acids (usually a linear epitope) or noncontiguous amino acids juxtaposed by tertiary folding of a protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation.

The term “binds to the same epitope” with reference to two or more antigen-binding moieties means that the antigen-binding moieties bind to the same segment of amino acid residues. Antigen-binding moieties that “compete with another antibody for binding to a target” refer to antigen-binding moieties that inhibit (partially or completely) the binding of the other antibody to the target.

As used herein, the terms “specific binding,” “selective binding,” “selectively binds,” and “specifically binds,” refer to an antigen-binding moiety (e.g., a CAR or an antibody) binding to an epitope on a predetermined antigen. Typically, the antigen-binding moiety (e.g., a CAR or an antibody) (i) binds with an equilibrium dissociation constant (K_{D}) of approximately less than 10⁻⁷ M, such as approximately less than 10⁻⁸ M, 10⁻⁹ M or 10⁻¹⁰ M or even lower when determined by, e.g., surface plasmon resonance (SPR) technology in a BIACORE® 2000 instrument using the predetermined antigen, e.g., human STEAP2, as the analyte and the antibody as the ligand, or Scatchard analysis of binding of the antibody to antigen positive cells, and (ii) binds to the predetermined antigen with an affinity that is at least two-fold greater than its affinity for binding to a non-specific antigen (e.g., BSA, casein) other than the predetermined antigen or a closely-related antigen. Accordingly, an antigen-binding moiety (e.g., a CAR or an antibody) that “specifically binds to human STEAP2” refers to an antigen-binding moiety (e.g., a CAR or an antibody) that binds to human STEAP2 with a K_{D} of 10⁻⁷ M or less, such as approximately less than 10⁻⁸ M, 10⁻⁹ M or 10⁻¹⁰ M or even lower.

The term “polypeptide,” as used herein, is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with, any of these terms. The term further includes polypeptides that have undergone post-translational or post-synthesis modifications, for example, conjugation of a palmitoyl group, glycosylation, acetylation, phosphorylation, amidation, derivatization by known protecting/blocking groups, disulfide bond formation, proteolytic cleavage, or modification by non-naturally occurring amino acids. The term “peptide,” as used herein, encompasses full-length peptides and fragments, variants or derivatives thereof. A “peptide” as used herein can be part of a fusion polypeptide comprising additional components such as, e.g., an albumin or PEG moiety, to increase half-life. A peptide as used herein can also be derivatized in a number of different ways. A peptide can comprise modifications including e.g., conjugation of a palmitoyl group. The term “nucleic acid molecule,” as used herein, is intended to include DNA molecules and RNA molecules. A nucleic acid molecule can be single-stranded or double-stranded, and can be cDNA.

“Conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in a STEAP2-binding moiety (e.g., an anti-STEAP2 CAR or antibody) is replaced with another amino acid residue from the same side chain family.

The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4: 11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

The term “promoter,” as used herein, refers to a DNA sequence recognized by the machinery of a cell, or introduced synthetic machinery, required to initiate the specific transcription of a gene. The term “promoter” is also meant to encompass those nucleic acid elements sufficient for promoter-dependent gene expression controllable for cell-type specific, tissue-specific or inducible expression by external signals or agents; such elements can be located in the 5′ or 3′ regions of the native gene. In some aspects, the promoter can be a constitutively active promoter, a cell-type specific promoter, or an inducible promoter.

The term “IRES,” as used herein, refers to an element that promotes direct internal ribosome entry to the initiation codon, such as ATG, of a cistron (a protein encoding region), thereby leading to the cap-independent translation of the gene. See, e.g., Jackson R J et al., Trends Biochem Sci 15(12):477-83 (199); Jackson R J and Kaminski, A. RNA 1(10):985-1000 (1995). Under translational control of an IRES translation proceeds in a cap-independent manner.

The term “termination signal sequence,” as used herein, can be any genetic element that causes RNA polymerase to terminate transcription, such as for example a polyadenylation signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

The terms “operatively linked,” “operatively inserted,” “operatively positioned,” “under control” or “under transcriptional control,” as used herein, mean that the promoter is in the correct location and orientation in relation to the nucleic acid to control RNA polymerase initiation and expression of the gene. The term “operably linked” means that a DNA sequence and a regulatory sequence(s) are connected in such a way as to permit gene expression when the appropriate molecules (e.g., transcriptional activator proteins) are bound to the regulatory sequence(s). The term “operably inserted” means that the DNA of interest introduced into a cell is positioned adjacent a DNA sequence which directs transcription and translation of the introduced DNA (i.e., facilitates the production of, e.g., a polypeptide encoded by a DNA of interest).

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, also included are other forms of expression vectors, such as viral vectors (e.g., replication defective retroviruses, adenoviruses and adeno-associated viruses), which serve equivalent functions.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because certain modifications can occur in succeeding generations due to either mutation or environmental influences, such progeny cannot, in fact, be identical to the parent cell, but are still included within the scope of the term “host cell” as used herein.

An “immune response” is as understood in the art, and generally refers to a biological response within a vertebrate against foreign agents or abnormal, e.g., cancerous cells, which response protects the organism against these agents and diseases caused by them. An immune response is mediated by the action of one or more cells of the immune system (for example, a T lymphocyte, B lymphocyte, natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the vertebrate's body of invading pathogens, cells or tissues infected with pathogens, cancerous or other abnormal cells, or, in cases of autoimmunity or pathological inflammation, normal human cells or tissues. An immune reaction includes, e.g., activation or inhibition of a T cell, e.g., an effector T cell, a Th cell, a CD4⁺ cell, a CD8⁺ T cell, or a Treg cell, or activation or inhibition of any other cell of the immune system, e.g., NK cell.

The term “lymphocyte” as used herein includes natural killer (NK) cells, T cells, or B cells. NK cells are a type of cytotoxic (cell toxic) lymphocyte that represent a major component of the inherent immune system. NK cells reject tumors and cells infected by viruses by inducing apoptosis or programmed cell death in the target cell. They were termed “natural killers” because NK cells do not require activation in order to kill a target cell. T-cells play a major role in cell-mediated-immunity. T-cell receptors (TCR) expressed on the surface of T cells differentiate T cells from other lymphocyte types. The thymus, a specialized organ of the immune system, is primarily responsible for T cell maturation. There are six types of T-cells, namely: Helper T-cells (e.g. CD4+ cells); Cytotoxic T-cells (also known as TC, cytotoxic T lymphocyte, CTL, T-killer cell, cytolytic T cell, CD8+ T-cells or killer T cell); Memory T-cells ((i) stem memory TSCM cells, like naive cells, are CD45RO−, CCR7+, CD45RA+, CD62L+ (L-selectin), CD27+, CD28+ and IL-7Ra+, but they also express large amounts of CD95, IL-2R.p, CXCR3, and LFA-1, and show numerous functional attributes distinctive of memory cells); (ii) central memory TCM cells express L-selectin and the CCR7, they secrete IL-2, but not IFNy or IL-4, and (iii) effector memory TEM cells, however, do not express L-selectin or CCR7 but produce effector cytokines like IFNy and IL-4); Regulatory T-cells (Tregs, suppressor T cells, or CD4+CD25+ regulatory T cells); Natural Killer T-cells (NKT); and Gamma Delta T-cells.

“Immunotherapy” refers to the treatment of a subject afflicted with, or at risk of contracting or suffering a recurrence of, a disease by a method comprising inducing, enhancing, suppressing or otherwise modifying the immune system or an immune response.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

As used herein, the terms “treat,” “treatment,” or “treatment of” when used in the context of treating cancer refer to reducing disease pathology, reducing or eliminating disease symptoms, promoting increased survival rates, and/or reducing discomfort. For example, treating can refer to the ability of a therapy when administered to a subject, to reduce disease symptoms, signs, or causes. Treating also refers to mitigating or decreasing at least one clinical symptom and/or inhibition or delay in the progression of the condition and/or prevention or delay of the onset of a disease or illness.

As used herein, “cancer” refers a broad group of diseases characterized by the uncontrolled growth of abnormal cells in the body. Unregulated cell division can result in the formation of malignant tumors or cells that invade neighboring tissues and can metastasize to distant parts of the body through the lymphatic system or bloodstream.

The terms “effective amount,” “therapeutically effective amount,” and a “sufficient amount” of, e.g., a CAR T cell and at least one androgen receptor antagonist or a composition described herein refer to a quantity sufficient to, when administered to a subject including a human effect beneficial or desired results, including alleviation of symptoms; diminishment of the extent of a condition, disorder, or disease; attainment of a stabilized (i.e., not worsening) state of a condition, disorder, or disease; delay in onset or slowing of a condition, disorder, or disease progression; amelioration of a condition, disorder, or disease state or remission (whether partial or total), whether detectable or undetectable; amelioration of at least one measurable physical parameter, not necessarily discernible by a patient; or enhancement or improvement of a condition, disorder, or disease. In some aspects, treatment includes eliciting a clinically significant response without excessive levels of side effects. As such, a “therapeutically effective amount” or synonyms thereof depend on the context in which they are applied. In some aspects, a therapeutically effective amount of an agent (e.g., a T cell engaging molecule or a composition described herein) is an amount that results in a beneficial or desired result in a subject as compared to a control that does not receive the agent. The amount of a given agent (e.g., a CAR T cell and at least one androgen receptor antagonist or a composition described herein) will vary depending upon various factors, such as the given agent, the pharmaceutical formulation, the route of administration, the type of disease or disorder, the identity of the subject (e.g., age, sex, and/or weight) or host being treated, and the like.

As used herein, the terms “subject,” “individual,” or “patient,” refer to any subject, particularly a mammalian subject, for whom diagnosis, prognosis, or therapy is desired. Mammalian subjects include, for example, humans, non-human primates, dogs, cats, guinea pigs, rabbits, rats, mice, horses, cattle, bears, and so on.

“Control” or “control experiment” or “standard control” is used in accordance with its plain ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects. In aspects, a control is an identical experiment or identical conditions without administration of a compound (e.g. a compound described herein). In aspects, inhibition of an activity compared to a control is inhibition of an activity by a compound (e.g., as described herein) compared to the activity in the absence of the compound (e.g. as described herein).

“Contacting” is used in accordance with its plain ordinary meaning and refers to the process of allowing at least two distinct species (e.g. chemical compounds including biomolecules, or cells) to become sufficiently proximal to react, interact or physically touch. It should be appreciated, however, that the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be a compound as described herein and a protein or enzyme. In some aspects contacting includes allowing a compound described herein to interact with a protein or enzyme.

As used herein, the term “administering” means oral administration, administration as a suppository, topical contact, intravenous, parenteral, intraperitoneal, intramuscular, intralesional, intrathecal, intracranial, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies (e.g. anti-cancer agent). The compound of the invention can be administered alone or can be coadministered to the patient. Coadministration is meant to include simultaneous or sequential administration of the compound individually or in combination (more than one compound or agent). Thus, the preparations can also be combined, when desired, with other active substances (e.g. to reduce metabolic degradation, to increase degradation of a prodrug and release of the drug, detectable agent). The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, dragees, capsules, liquids, lozenges, cachets, gels, syrups, slurries, suspensions, etc., suitable for ingestion by the patient. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In another aspect, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

As used herein, “survival” refers to the patient remaining alive, and includes overall survival as well as progression free survival. 1-year survival rate and 2-year survival rate refers to the K-M estimate of the proportion of subjects alive at 12 month or 24 months.

By “extending survival” is meant increasing overall survival and/or progression free survival in a treated patient relative to a control treatment protocol, such as treatment with the antibody drug conjugates described herein. Survival is monitored for at least about one month, two months, four months, six months, nine months, or at least about 1 year, or at least about 2 years, or at least about 3 years, or at least about 4 years, or at least about 5 years, or at least about 10 years, etc., following the initiation of treatment or following the initial diagnosis.

By “reduce or inhibit” is meant the ability to cause an overall decrease of 20%, 30%, 40%, 50%, 60%, 70%, 75%, 80%, 85%, 90%, 95%, or greater. Reduce or inhibit can refer to the symptoms of the disorder being treated, the presence or size of metastases, or the size of the primary tumor.

As used herein, the term “inhibition”, “inhibit”, “inhibiting” and the like in reference to cell proliferation (e.g., cancer cell proliferation) means negatively affecting (e.g., decreasing proliferation) or killing the cell. In some aspects, inhibition refers to reduction of a disease or symptoms of disease (e.g., cancer, cancer cell proliferation). Thus, inhibition includes, at least in part, partially or totally blocking stimulation, decreasing, preventing, or delaying activation, or inactivating, desensitizing, or down-regulating signal transduction or enzymatic activity or the amount of a protein. Similarly an “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

The term “pharmaceutically acceptable salts” is meant to include salts of the active compounds that are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al. Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention. Salts tend to be more soluble in aqueous or other protonic solvents than are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

As used herein, the terms “ug” and “uM” are used interchangeably with “μg” and “μM,” respectively.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

Various aspects described herein are described in further detail in the following subsections.

### I. Methods of the Disclosure

The chimeric antigen receptor (CAR) T cells and androgen receptor antagonists described herein may be used for therapeutic application, such as in the treatment of cancer, e.g., prostate cancer.

In some aspects, a CAR T cell and at least one androgen receptor antagonist as described herein may be used in a method of treatment of the human or animal body. Related aspects of the disclosure provide:
(i) a CAR T cell and at least one androgen receptor antagonist as described herein for use as a medicament;
(ii) a CAR T cell and at least one androgen receptor antagonist as described herein for use in a method of treatment of a disease or disorder;
(iii) a method for inhibiting the growth of a tumor cell, wherein the method comprises contacting the tumor cell with a CAR T cell and at least one androgen receptor antagonist as described herein;
(iv) a CAR T cell and androgen receptor antagonist as described herein in the manufacture of a medicament for use in the treatment of a disease or disorder; and,
(v) a method of treating a disease or disorder in a subject, wherein the method comprises administering to the subject in need thereof a therapeutically effective amount of a CAR T cell and androgen receptor antagonist as described herein.

The subject may be a patient, preferably a human patient, e.g. a patient suffering from prostate cancer.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment. In some aspects, the method is a method of treating cancer.

Treatment as a prophylactic measure (i.e. prophylaxis) is also included. For example, an individual susceptible to or at risk of the occurrence or re-occurrence of a disease such as cancer may be treated as described herein. Such treatment may prevent or delay the occurrence or re-occurrence of the disease in the individual.

Some aspects of the present disclosure are directed to a method for inhibiting the growth of a tumor cell comprising contacting said tumor cell with: a T cell comprising (i) a polynucleotide encoding a chimeric antigen receptor (CAR) that binds an epitope on human six transmembrane epithelial antigen of prostate-2 (STEAP2); and at least one androgen receptor antagonist, in an amount effective to inhibit tumor cell growth. In some aspects, the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

Some aspects of the present disclosure are directed to a method for inhibiting the growth of a tumor cell comprising contacting said tumor cell with: a T cell comprising (i) a polynucleotide encoding a chimeric antigen receptor (CAR) that binds an epitope on human six transmembrane epithelial antigen of prostate-2 (STEAP2); and at least one androgen receptor antagonist, in an amount effective to inhibit tumor cell growth; wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

Some aspects of the present disclosure are direct to a method for treating a cancer comprising tumor cells in a subject comprising administering to said subject in need thereof a therapeutically effective amount of: a T cell comprising (i) a polynucleotide encoding a chimeric antigen receptor (CAR) that binds an epitope on human six transmembrane epithelial antigen of prostate-2 (STEAP2); and at least one androgen receptor antagonist. In some aspects, the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

Some aspects of the present disclosure are direct to a method for treating a cancer comprising tumor cells in a subject comprising administering to said subject in need thereof a therapeutically effective amount of: a T cell comprising (i) a polynucleotide encoding a chimeric antigen receptor (CAR) that binds an epitope on human six transmembrane epithelial antigen of prostate-2 (STEAP2); and at least one androgen receptor antagonist; wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

In some aspects, the VH comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and the VL comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8.

In some aspects, the VH comprises the amino acid sequence set forth in SEQ ID NO: 7, and the VL comprises the amino acid sequence set forth in SEQ ID NO: 8.

In some aspects, the polynucleotide further encodes an armoring molecule and said armoring molecule comprises a dominant-negative TGF-β receptor type 2 (TGFβRIIDN).

In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10.

In some aspects, the armoring molecule comprises the amino acid sequence set forth in SEQ ID NO: 10.

In some aspects, the polynucleotide encoding the CAR comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 11.

In some aspects, the polynucleotide encoding the CAR comprises the nucleotide sequence set forth in SEQ ID NO: 11.

In some aspects, the polynucleotide encoding the CAR comprises the nucleotide sequence set forth in SEQ ID NO: 11, and the polynucleotide encoding the armoring molecule comprises the nucleotide sequence set forth in SEQ ID NO: 9.

In some aspects, the polynucleotide encoding the CAR and the polynucleotide encoding the armoring molecule are operably linked under the control of a single promoter.

In some aspects, the polynucleotide encoding the CAR and the polynucleotide encoding the armoring molecule are operably linked by an IRES.

In some aspects, the polynucleotide encoding the CAR and the polynucleotide encoding the armoring molecule are linked by a nucleotide sequence encoding a cleavable peptide linker. In some aspects, the cleavable peptide linker is a self-cleaving peptide linker. In some aspects, the cleavable peptide linker comprises a T2A peptide. In some aspects, the cleavable peptide linker comprises SEQ ID NO: 13.

In some aspects, the polynucleotide comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 14.

In some aspects, the polynucleotide comprises the nucleotide sequence set forth in SEQ ID NO: 14.

In some aspects, the T cell comprises an amino acid sequence that comprises the amino acid sequence set forth in SEQ ID NO: 12.

In some aspects, the T cell comprises an amino acid sequence that comprises the amino acid sequence set forth in SEQ ID NO: 15.

In some aspects, the androgen receptor antagonist is enzalutamide, apalutamide, darolutamide, abiraterone, bicalutamide, nilutamide, flutamide, proxalutamide, or a combination thereof.

In some aspects, the androgen receptor antagonist is enzalutamide.

In some aspects, the androgen receptor antagonist is abiraterone.

In some aspects, the method comprises administering two or more androgen receptor antagonists. In some aspects, the method comprises administering two androgen receptor antagonists. In some aspects, the method comprises administering enzalutamide and a different androgen receptor antagonist. In some aspects, the method comprises administering abiraterone and a different androgen receptor antagonist. In some aspects, the method comprises administering abiraterone and enzalutamide.

In some aspects, the method further comprises administering at least one further treatment to the subject in addition to the CAR T cell and at least one androgen receptor antagonist. The CAR T cell and at least one androgen receptor antagonist described herein may thus be administered to a subject subject alone or in combination with one or more other treatments. Where the CAR T cell and at least one androgen receptor antagonist is administered to the subject in combination with another treatment, the additional treatment may be administered to the individual concurrently with, sequentially to, or separately from the administration of the CAR T cell and at least one androgen receptor antagonist. Where the additional treatment is administered concurrently with the CAR T cell and at least one androgen receptor antagonist, the CAR T cell, at least one androgen receptor antagonist, and additional treatment may be administered to the subject as a combined preparation. For example, the additional therapy may be a known therapy or therapeutic agent for the disease to be treated, e.g., prostate cancer.

In some aspects, the CAR T cell and/or at least one androgen receptor antagonist are administered in the form of a pharmaceutical composition, which may comprise at least one component in addition to the CAR T cell and/or at least one androgen receptor antagonist. Another aspect of the invention therefore provides a pharmaceutical composition comprising a CAR T cell and at least one androgen receptor antagonistas described herein. A method comprising formulating a CAR T cell and/or at least one androgen receptor antagonist into a pharmaceutical composition is also provided.

Pharmaceutical compositions may comprise, in addition to the CAR T cell and/or at least one androgen receptor antagonist, a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

In some aspects, a CAR T cell and/or at least one androgen receptor antagonist may be provided in a lyophilized form for reconstitution prior to administration. For example, lyophilized CAR T cells and/or at least one androgen receptor antagonist may be re-constituted in sterile water and mixed with saline prior to administration to a subject.

Administration may be in a “therapeutically effective amount”, this being sufficient to show benefit in a subject. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, the scheduling of administration and other factors known to medical practitioners.

In some aspects, the androgen receptor antagonist is administered orally.

In some aspects, the T cell is administered intravenously.

In some aspects, the T cell and the androgen receptor antagonist are administered sequentially or simultaneously, and in any order.

In some aspects, administration of the androgen receptor antagonist does not inhibit T cell viability or T cell killing capacity.

In some aspects, administration of the androgen receptor antagonist does not inhibit interferon-gamma (IFNγ) release in tumor cells.

In some aspects, administration of the androgen receptor antagonist does not reduce CAR expression on the T cell.

In some aspects, the T cell is a CD8+ T cell.

In some aspects, the tumor cells are prostate tumor cells, optionally wherein the prostate tumor cells are metastatic, recurrent, or relapsed.

In some aspects, the administration increases STEAP2 expression in the tumor cells as compared to the expression of STEAP2 in the tumor cells when the T-cell is administered alone. In some aspects, the administration increases STEAP2 expression by from about 50% to about 200% in the tumor cells as compared to the expression of STEAP2 in the tumor cells when the T-cell is administered alone. In some aspects, the STEAP2 expression is increased by about 100%.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect and exposure in association with the required pharmaceutical carrier. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

Some aspects of the present disclosure are directed to a use of a T cell and an androgen receptor antagonist in a method for inhibiting the growth of a tumor cell, the method comprising contacting said tumor cell with the T cell and at least one androgen receptor antagonist, in an amount effective to inhibit tumor cell growth, wherein the T cell comprises (i) a polynucleotide encoding a CAR that binds an epitope on human STEAP2 and (ii) a polynucleotide encoding an armoring molecule. In some aspects, the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6. In some aspects, the tumor cells are prostate tumor cells. In some aspects, the prostate tumor cells are metastatic, recurrent, or relapsed.

Some aspects of the present disclosure are directed to a use of a T cell and an androgen receptor antagonist in a method for inhibiting the growth of a tumor cell, the method comprising contacting said tumor cell with the T cell and at least one androgen receptor antagonist, in an amount effective to inhibit tumor cell growth, wherein the T cell comprises (i) a polynucleotide encoding a CAR that binds an epitope on human STEAP2 and (ii) a polynucleotide encoding an armoring molecule, wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6. In some aspects, the tumor cells are prostate tumor cells. In some aspects, the prostate tumor cells are metastatic, recurrent, or relapsed.

Some aspects of the present disclosure are directed to a use of a T cell and an androgen receptor antagonist in a method of treating a cancer comprising tumor cells in a subject comprising administering to said subject in need thereof a therapeutically effective amount of the T cell and at least one androgen receptor antagonist, wherein the T cell comprises (i) a polynucleotide encoding a CAR that binds an epitope on human STEAP2 and (ii) a polynucleotide encoding an armoring molecule. In some aspects, the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6. In some aspects, the tumor cells are prostate tumor cells. In some aspects, the prostate tumor cells are metastatic, recurrent, or relapsed.

Some aspects of the present disclosure are directed to a use of a T cell and an androgen receptor antagonist in a method of treating a cancer comprising tumor cells in a subject comprising administering to said subject in need thereof a therapeutically effective amount of the T cell and at least one androgen receptor antagonist, wherein the T cell comprises (i) a polynucleotide encoding a CAR that binds an epitope on human STEAP2 and (ii) a polynucleotide encoding an armoring molecule, wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6. In some aspects, the tumor cells are prostate tumor cells. In some aspects, the prostate tumor cells are metastatic, recurrent, or relapsed.

Certain aspects of the present disclosure are directed to methods of treating a disease or condition in a subject in need thereof, comprising administering to the subject a composition disclosed herein. In some aspects, the disease or condition comprises a cancer. In some aspects, the cancer is prostate cancer. In some aspects, the cancer comprises a tumor derived from a prostate cancer (e.g., a tumor arising from the metastasis of a prostate cancer). In some aspects, the cancer (e.g., the prostate cancer) is locally progressed. In some aspects, the cancer (e.g., the prostate cancer) is metastatic. In some aspects, the cancer (e.g., the prostate cancer) is recurrent. In some aspects, the cancer (e.g., the prostate cancer) is relapsed.

The compositions disclosed herein, e.g. a T-cell comprising a polynucleotide encoding a CAR disclosed herein, can be used in combination with other anti-cancer therapies, including one or more additional immunotherapies. In some aspects, the compositions disclosed herein are administered concurrently with the additional anti-cancer agent. In some aspects, the compositions disclosed herein and the additional anti-cancer agent are administered sequentially (e.g., on the same day or on different days).

In various aspects, the disclosed anti-STEAP2 CAR T cell and at least one androgen receptor antagonist may be administered in any cell or tissue that expresses STEAP2, such as a STEAP2-expressing neoplastic cell or tissue. An exemplary aspect includes a method of inhibiting STEAP2-mediated cell signaling or a method of killing a cell. The method may be used with any cell or tissue that expresses STEAP2, such as a cancerous cell or a metastatic lesion. Non-limiting examples of STEAP2-expressing cancers include prostate cancer, bladder cancer, colon cancer, pancreatic cancer, ovarian cancer, testicular cancer, Ewing's sarcoma, or cervical cancer.

Exemplary methods include the steps of contacting a cell with a CAR T cell and at least one androgen receptor antagonist, as described herein, in an effective amount, i.e., amount sufficient to kill the cell. The method can be used on cells in culture, e.g. in vitro, in vivo, ex vivo, or in situ. For example, cells that express STEAP2 (e.g., cells collected by biopsy of a tumor or metastatic lesion; cells from an established cancer cell line; or recombinant cells), can be cultured in vitro in culture medium and the contacting step can be affected by adding the CAR T cell and at least one androgen receptor antagonist to the culture medium. The method will result in killing of cells expressing STEAP2, including in particular tumor cells expressing STEAP2. Alternatively, the CAR T cell and at least one androgen receptor antagonist can be administered to a subject by any suitable administration route (e.g., intravenous, subcutaneous, or direct contact with a tumor tissue) to have an effect in vivo.

The in vivo effect of a disclosed CAR T cell and androgen receptor antagonist therapeutic composition can be evaluated in a suitable animal model. For example, xenogeneic cancer models can be used, wherein cancer explants or passaged xenograft tissues are introduced into immune compromised animals, such as nude or SCID mice (Klein et al. (1997) Nature Med. 3:402-8). Patient-derived xenografts (PDX), in which tumor tissues from patients are implanted into immunocompromised or humanized mice (see, e.g., Liu Y et al. Signal Transduct. Target Ther. 2023; 8(1):160), may also be used to evaluate the in vivo effect of a disclosed CAR T cell and androgen receptor antagonist therapeutic composition. Efficacy may be predicted using assays that measure inhibition of tumor formation, tumor regression or metastasis, and the like.

For determining cytotoxicity, necrosis or apoptosis (programmed cell death) may be measured. Necrosis is typically accompanied by increased permeability of the plasma membrane; swelling of the cell, and rupture of the plasma membrane. Apoptosis can be quantitated, for example, by measuring DNA fragmentation. Commercial photometric methods for the quantitative in vitro determination of DNA fragmentation are available. Examples of such assays, including TUNEL (which detects incorporation of labeled nucleotides in fragmented DNA) and ELISA-based assays, are described in Biochemica (1999) No. 2, pp. 34-37 (Roche Molecular Biochemicals).

Apoptosis may also be determined by measuring morphological changes in a cell. For example, as with necrosis, loss of plasma membrane integrity can be determined by measuring uptake of certain dyes (e.g., a fluorescent dye such as, for example, acridine orange or ethidium bromide). A method for measuring apoptotic cell number has been described by Duke and Cohen, Current Protocols in Immunology (Coligan et al., eds. (1992) pp. 3.17.1-3.17.16). Cells also can be labeled with a DNA dye (e.g., acridine orange, ethidium bromide, or propidium iodide) and the cells observed for chromatin condensation and margination along the inner nuclear membrane. Apoptosis may also be determined, in some aspects, by screening for caspase activity. In some aspects, a Caspase-Glo® Assay can be used to measure activity of caspase-3 and caspase-7. In some aspects, the assay provides a luminogenic caspase-3/7 substrate in a reagent optimized for caspase activity, luciferase activity, and cell lysis. In some aspects, adding Caspase-Glo® 3/7 Reagent in an “add-mix-measure” format may result in cell lysis, followed by caspase cleavage of the substrate and generation of a “glow-type” luminescent signal, produced by luciferase. In some aspects, luminescence may be proportional to the amount of caspase activity present, and can serve as an indicator of apoptosis. Other morphological changes that can be measured to determine apoptosis include, e.g., cytoplasmic condensation, increased membrane blebbing, and cellular shrinkage. Determination of any of these effects on cancer cells indicates that an ADC is useful in the treatment of cancers.

Cell viability may be measured, e.g., by determining in a cell the uptake of a dye such as neutral red, trypan blue, Crystal Violet, or ALAMAR™ blue (see, e.g., Page et al. (1993) Intl J Oncology 3:473-6). In such an assay, the cells are incubated in media containing the dye, the cells are washed, and the remaining dye, reflecting cellular uptake of the dye, is measured spectrophotometrically. Cell viability may also be measured, e.g., by quantifying ATP, an indicator of metabolically active cells. In certain aspects, in vitro potency and/or cell viability of prepared ADCs may be assessed using a CellTiter-Glo® Luminescent Cell Viability Assay, as described in the examples provided herein. In this assay, in certain aspects, the single reagent (CellTiter-Glo® Reagent) is added directly to cells cultured in serum-supplemented medium. The addition of reagent results in cell lysis and generation of a luminescent signal proportional to the amount of ATP present. The amount of ATP is directly proportional to the number of cells present in culture. The protein-binding dye sulforhodamine B (SRB) can also be used to measure cytotoxicity (Skehan et al. (1990) J Natl Cancer Inst. 82:1107-12).

In vivo assays that evaluate the promotion of tumor cell death and tumor schrinkage by mechanisms such as apoptosis may also be used. In one aspect, xenografts from tumor bearing mice treated with the therapeutic composition can be examined for the presence of apoptotic foci and compared to untreated control xenograft-bearing mice. The extent to which apoptotic foci are found in the tumors of the treated mice provides an indication of the therapeutic efficacy of the composition.

An exemplary aspect is a method of reducing or inhibiting growth of a tumor (e.g., a STEAP2-expressing tumor), comprising administering a therapeutically effective amount of a CAR T cell and at least one androgen receptor antagonist. In some aspects, the treatment is sufficient to reduce or inhibit the growth of the patient's tumor, reduce the number or size of metastatic lesions, reduce tumor load, reduce primary tumor load, reduce invasiveness, prolong survival time, and/or maintain or improve the quality of life. In some aspects, the tumor is resistant or refractory to treatment with a CAR T cell (e.g., an anti-STEAP2 CAR T cell) when administered alone.

In various aspects, treatment involves single bolus or repeated administration of the CAR T cell and at least one androgen receptor antagonist preparation via an acceptable route of administration.

Patients may be evaluated for the levels of target antigen in a given sample (e.g. the levels of target antigen expressing cells) in order to assist in determining the most effective dosing regimen, etc. An exemplary aspect is a method of determining whether a patient will be responsive to treatment with a CAR T cell and at least one androgen receptor antagonist of the present disclosure, comprising providing a biological sample from the patient and contacting the biological sample with the CAR T cell and at least one androgen receptor antagonist. Exemplary biological samples include tissue, stool sample, or tumor biopsy (e.g., a tumor biopsy derived from a patient having or at risk of a target antigen-expressing cancer, e.g., a STEAP2-expressing cancer). In some aspects, a sample (e.g., a tissue) can be obtained from a subject, and a suitable immunological method can be used to detect and/or measure protein expression of the target antigen (e.g., STEAP2). Such evaluations are also used for monitoring purposes throughout therapy, and are useful to gauge therapeutic success in combination with the evaluation of other parameters.

In some aspects, the efficacy of a CAR T cell and at least one androgen receptor antagonist may be evaluated by contacting a tumor sample from a subject with the CAR T cell and at least one androgen receptor antagonist and evaluating tumor growth rate or volume. In some aspects, when a CAR T cell and at least one androgen receptor antagonist has been determined to be effective, it may be administered to the subject.

In some aspects, co-administration includes administering one active agent (e.g., a CAR T cell) within 0.5, 1, 2, 4, 6, 8, 10, 12, 16, 20, or 24 hours of at least one androgen receptor antagonist, in either order. Co-administration also includes administering two active agents simultaneously, approximately simultaneously (e.g., within about 1, 5, 10, 15, 20, or 30 minutes of each other), or sequentially, in any order. In some aspects, co-administration can be accomplished by co-formulation, i.e., preparing a single pharmaceutical composition including both active agents. In other aspects, the active agents can be formulated separately. In another aspect, the active and/or adjunctive agents may be linked or conjugated to one another. In some aspects, the compounds described herein may be combined with treatments for cancer such as radiation or surgery.

Specific in vivo clinical protocols with regard to route of administration, excipients, diluents, dosages, times, etc., can be determined by one of ordinary skill in the art as the clinical situation warrants. The dosage and frequency (single or multiple doses) administered to a mammal can vary depending upon a variety of factors, for example, whether the mammal suffers from another disease, and its route of administration; size, age, sex, health, body weight, body mass index, and diet of the recipient; nature and extent of symptoms of the disease being treated (e.g. symptoms of cancer), kind of concurrent treatment, complications from the disease being treated or other health-related problems. Other therapeutic regimens or agents can be used in conjunction with the methods and compounds of Applicants' invention. Adjustment and manipulation of established dosages (e.g., frequency and duration) are well within the ability of those skilled in the art.

### II. Polynucleotides of the Disclosure

Some aspects of the present disclosure are directed to polynucleotides comprising a nucleotide sequence encoding a CAR that specifically binds human STEAP2. In some aspects, the CAR comprises (i) an antigen-binding domain that binds an epitope on STEAP2, (ii) a transmembrane domain, and (iii) an intracellular domain. In some aspects, the CAR further comprises a hinge/spacer domain. In some aspects, the hinge/spacer domain is positioned between the antigen-binding domain and the transmembrane domain.

In some aspects, the polynucleotide further comprises a nucleotide sequence encoding an armoring molecule. In some aspects, the nucleotide sequence encoding the CAR and the nucleotide sequence encoding the armoring moiety are expressed under the control of the same promoter. In some aspects, the nucleotide sequence encoding the CAR and the nucleotide sequence encoding the armoring moiety are expressed under the control of two promoter. In some aspects, the two promoters are different promoters. In some aspects, the nucleotide sequence encoding the CAR and the nucleotide sequence encoding the armoring moiety are expressed as a single contiguous polypeptide. In some aspects, the nucleotide sequence encoding the CAR and the nucleotide sequence encoding the armoring moiety are expressed as two separate polypeptides. In some aspects, the CAR and the nucleotide sequence encoding the armoring moiety are linked by a nucleotide sequence encoding a linker. In some aspects, the linker is a peptide linker. In some aspects, the linker is a cleavable linker. In some aspects, the linker is a self-cleaving peptide linker, e.g., comprising a T2A peptide.

### I.A. Antigen-Binding Domain

Disclosed herein are polynucleotides comprising a nucleotide sequence encoding a CAR, wherein the CAR comprises (i) an antigen-binding domain that binds an epitope on human STEAP2, (ii) an intracellular signaling domain, and (iii) a transmembrane domain. Any antigen-binding domain can be used in the compositions disclosed herein. In some aspects, the antigen-binding domain comprises an Fab, Fab′, F(ab′)2, Fd, Fv, single-chain fragment variable (scFv), single chain antibody, VHH, vNAR, nanobody (single-domain antibody), or any combination thereof. In some aspects, the antigen-binding domain comprises a scFv.

In some aspects, the antigen-binding domain of the CAR comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3. In some aspects, the antigen-binding domain comprises a VH-CDR3 comprising an amino acid sequence selected from SEQ ID NOs: 6. In some aspects, the antigen-binding domain comprises a VH-CDR2 comprising an amino acid sequence selected from SEQ ID NOs: 5. In some aspects, the antigen-binding domain comprises a VH-CDR1 comprising an amino acid sequence selected from SEQ ID NOs: 4.

In some aspects, the antigen-binding domain comprises a VL-CDR3 comprising an amino acid sequence selected from SEQ ID NOs: 3. In some aspects, wherein the antigen-binding domain comprises a VL-CDR2 comprising an amino acid sequence selected from SEQ ID NOs: 2. In some aspects, the antigen-binding domain comprises a VL-CDR1 comprising an amino acid sequence selected from SEQ ID NOs: 1.

In some aspects, the antigen binding domain comprises a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7. In some aspects, the antigen-binding domain comprises a VH comprising an amino acid sequence selected from SEQ ID NOs: 7.

In some aspects, the CAR comprises an antigen-binding domain comprising a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8. In some aspects, the antigen-binding domain comprises a VL comprising an amino acid sequence selected from SEQ ID NOs: 8.

In some aspects, the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the CAR comprises an antigen-binding domain comprising a VH comprising the amino acid sequence set forth in SEQ ID NO: 7, and a VL comprising the amino acid sequence set forth in SEQ ID NO: 8.

In some aspects, the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, the CAR comprises an antigen-binding domain comprising the amino acid sequence set forth in SEQ ID NO: 16.

### I.B. Intracellular Domain

Disclosed herein are polynucleotides comprising a nucleotide sequence encoding a CAR, wherein the CAR comprises (i) an antigen-binding domain that binds an epitope on human STEAP2, (ii) an intracellular signaling domain, and (iii) a transmembrane domain. Any intracellular signaling domain can be used in the compositions disclosed herein. In some aspects, the intracellular signaling domain comprises a costimulatory domain or a portion thereof.

In some aspects, the intracellular domain comprises a costimulatory domain selected from the group consisting of the intracellular domain of CD3z, a CD28 co-stimulatory domain, a CD27 co-stimulatory domain, a 4-1BB co-stimulatory domain, an ICOS co-stimulatory domain, an OX-40 co-stimulatory domain, a GITR co-stimulatory domain, a CD2 co-stimulatory domain, an IL-2Rβ co-stimulatory domain, an MyD88/CD40a CD28 co-stimulatory domain, and any combination thereof.

In some aspects, the intracellular domain comprises a 4-1BB co-stimulatory domain. In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (ii) a 4-1BB costimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 130. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; and (ii) a 4-1BB co-stimulatory domain comprising the amino acid sequence set forth in SEQ ID NO: 17.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; and (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17. In some aspects, the CAR comprises (i) an antigen-binding domain comprising the amino acid sequence set forth in SEQ ID NO: 16; and (ii) a 4-1BB co-stimulatory domain comprising the amino acid sequence set forth in SEQ ID NO: 17.

In some aspects, the intracellular domain comprises the intracellular domain of CD3z and a CD28 co-stimulatory domain. In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (ii) the intracellular domain of CD3z and a CD28 co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; and (ii) the intracellular domain of CD3z comprising the amino acid sequence set forth in SEQ ID NO: 18 and a CD28 co-stimulatory domain comprising the amino acid sequence set forth in SEQ ID NO: 19.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19. In some aspects, the CAR comprises (i) an antigen-binding domain comprising the amino acid sequence set forth in SEQ ID NO: 16; and (ii) the intracellular domain of CD3z comprising the amino acid sequence set forth in SEQ ID NO: 18 and a CD28 co-stimulatory domain comprising the amino acid sequence set forth in SEQ ID NO: 19.

In some aspects, the intracellular domain comprises the intracellular domain of CD3z and a 4-1BB co-stimulatory domain. In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (ii) the intracellular domain of CD3z and a 4-1BB co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; and (ii) the intracellular domain of CD3z comprising the amino acid sequence set forth in SEQ ID NO: 18 and a 4-1BB co-stimulatory domain comprising the amino acid sequence set forth in SEQ ID NO: 17.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17. In some aspects, the CAR comprises (i) an antigen-binding domain comprising the amino acid sequence set forth in SEQ ID NO: 16; and (ii) the intracellular domain of CD3z comprising the amino acid sequence set forth in SEQ ID NO: 18 and a 4-1BB co-stimulatory domain: comprising the amino acid sequence set forth in SEQ ID NO: 17.

In some aspects, the intracellular domain comprises the intracellular domain of CD3z, a CD28 co-stimulatory domain, and a 4-1BB co-stimulatory domain. In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (ii) the intracellular domain of CD3z, a CD28 co-stimulatory domain, and a 4-1BB co-stimulatory domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; and (ii) the intracellular domain of CD3z comprising the amino acid sequence set forth in SEQ ID NO: 18, a CD28 co-stimulatory domain comprising the amino acid sequence set forth in SEQ ID NO: 19, and a 4-1BB co-stimulatory domain comprising the amino acid sequence set forth in SEQ ID NO: 17.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; and (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17. In some aspects, the CAR comprises (i) an antigen-binding domain comprising the amino acid sequence set forth in SEQ ID NO: 16; and (ii) the intracellular domain of CD3z comprising the amino acid sequence set forth in SEQ ID NO: 18, a CD28 co-stimulatory domain comprising the amino acid sequence set forth in SEQ ID NO: 19, and a 4-1BB co-stimulatory domain comprising the amino acid sequence set forth in SEQ ID NO: 17.

In some aspects, the CAR comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 12. In some aspects, the CAR comprises the amino acid sequence set forth in SEQ ID NO: 12.

### I.C. Transmembrane Domain

Disclosed herein are polynucleotides comprising a nucleotide sequence encoding a CAR, wherein the CAR comprises (i) an antigen-binding domain that binds an epitope on human STEAP2, (ii) an intracellular signaling domain, and (iii) a transmembrane domain. Any transmembrane domain can be used in the compositions disclosed herein. In some aspects, the transmembrane domain comprises a transmembrane domain selected from the transmembrane domain of CD4, CD8α, or CD28. In some aspects, the transmembrane domain comprises a CD28 transmembrane domain.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) a 4-1BB costimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) a 4-1BB co-stimulatory domain comprising the amino acid sequence set forth in SEQ ID NO: 17; and (iii) a transmembrane domain comprising the amino acid sequence set forth in SEQ ID NO: 20.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the CAR comprises (i) an antigen-binding domain comprising the amino acid sequence set forth in SEQ ID NO: 16; (ii) a 4-1BB co-stimulatory domain comprising the amino acid sequence set forth in SEQ ID NO: 17; and (iii) a transmembrane domain comprising the amino acid sequence set forth in SEQ ID NO: 20.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z and a CD28 co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z comprising the amino acid sequence set forth in SEQ ID NO: 18 and a CD28 co-stimulatory domain comprising the amino acid sequence set forth in SEQ ID NO: 19; and (iii) a transmembrane domain comprising the amino acid sequence set forth in SEQ ID NO: 20.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19; and (iii) a transmembrane domain comprising the transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the CAR comprises (i) an antigen-binding domain comprising the amino acid sequence set forth in SEQ ID NO: 16; (ii) the intracellular domain of CD3z comprising the amino acid sequence set forth in SEQ ID NO: 18 and a CD28 co-stimulatory domain comprising the amino acid sequence set forth in SEQ ID NO: 19; and (iii) a transmembrane domain comprising the amino acid sequence set forth in SEQ ID NO: 20.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z and a 4-1BB co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; and (iii) a transmembrane domain comprising comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z comprising the amino acid sequence set forth in SEQ ID NO: 18 and a 4-1BB co-stimulatory domain comprising the amino acid sequence set forth in SEQ ID NO: 17; and (iii) a transmembrane domain comprising the amino acid sequence set forth in SEQ ID NO: 20.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO:17; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the CAR comprises (i) an antigen-binding domain comprising the amino acid sequence set forth in SEQ ID NO: 16; (ii) the intracellular domain of CD3z comprising the amino acid sequence set forth in SEQ ID NO: 18 and a 4-1BB co-stimulatory domain comprising the amino acid sequence set forth in SEQ ID NO: 17; and (iii) a transmembrane domain comprising the amino acid sequence set forth in SEQ ID NO: 20.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z, a CD28 co-stimulatory domain, and a 4-1BB co-stimulatory domain; and (iii) a transmembrane domain comprising the transmembrane domain of CD28. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z comprising the amino acid sequence set forth in SEQ ID NO: 18, a CD28 co-stimulatory domain comprising the amino acid sequence set forth in SEQ ID NO: 19, and a 4-1BB co-stimulatory domain comprising the amino acid sequence set forth in SEQ ID NO: 17; and (iii) a transmembrane domain comprising the amino acid sequence set forth in SEQ ID NO: 20.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; and (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20. In some aspects, the CAR comprises (i) an antigen-binding domain comprising the amino acid sequence set forth in SEQ ID NO: 16; (ii) the intracellular domain of CD3z comprising the amino acid sequence set forth in SEQ ID NO: 18, a CD28 co-stimulatory domain comprising the amino acid sequence set forth in SEQ ID NO: 19, and a 4-1BB co-stimulatory domain comprising the amino acid sequence set forth in SEQ ID NO: 17; and (iii) a transmembrane domain comprising comprising the amino acid sequence set forth in SEQ ID NO: 20.

In some aspects, the CAR comprises an amino acid sequence set forth in SEQ ID NO: 12.

### I.D. Spacer/Hinge Domain

Disclosed herein are polynucleotides comprising a nucleotide sequence encoding a CAR, wherein the CAR comprises (i) an antigen-binding domain that binds an epitope on human STEAP2, (ii) an intracellular signaling domain, (iii) a transmembrane domain, and (iv) a hinge/spacer domain. Any hinge/spacer domain can be used in the compositions disclosed herein. In some aspects, the hinge/spacer domain comprises a human immunoglobulin hinge/spacer domain. In some aspects, the hinge/spacer domain comprises an IgG hinge domain. In some aspects, the hinge/spacer domain comprise an IgG1 hinge domain, and IgG2 hinge domain, an IgG3 hinge domain, or an IgG4 hinge domain. In some aspects, the hinge/spacer domain comprises an IgG4 hinge domain. In some aspects, the IgG hinge domain is a variant hinge domain. In some aspects, the IgG4 hinge domain is a variant IgG4 hinge domain. In some aspects, the variant IgG4 hinge domain comprises a S228P mutation. In some aspects, the IgG4 hinge domain comprises an amino acid sequence set forth in SEQ ID NO: 21.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) a 4-1BB costimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20 and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) a 4-1BB co-stimulatory domain comprising the amino acid sequence set forth in SEQ ID NO: 17; (iii) a transmembrane domain comprising the amino acid sequence set forth in SEQ ID NO: 20; and (iv) an IgG hinge domain comprising the amino acid sequence set forth in SEQ ID NO: 21.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; (ii) a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the CAR comprises (i) an antigen-binding domain comprising the amino acid sequence set forth in SEQ ID NO: 16; (ii) a 4-1BB co-stimulatory domain comprising the amino acid sequence set forth in SEQ ID NO: 17; (iii) a transmembrane domain comprising the amino acid sequence set forth in SEQ ID NO: 20; and (iv) an IgG hinge domain comprising the amino acid sequence set forth in SEQ ID NO: 21.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z and a CD28 co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z comprising the amino acid sequence set forth in SEQ ID NO: 18 and a CD28 co-stimulatory domain comprising the amino acid sequence set forth in SEQ ID NO: 19; (iii) a transmembrane comprising the amino acid sequence set forth in SEQ ID NO: 20; and (iv) an IgG hinge domain comprising the amino acid sequence set forth in SEQ ID NO: 21.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the CAR comprises (i) an antigen-binding domain comprising the amino acid sequence set forth in SEQ ID NO: 16; (ii) the intracellular domain of CD3z and a CD28 co-stimulatory domain; (iii) a transmembrane domain comprising the transmembrane domain of CD28; and (iv) an IgG hinge domain.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z and a 4-1BB co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z and a 4-1BB co-stimulatory domain; (iii) a transmembrane domain comprising the transmembrane domain of CD28; and (iv) an IgG hinge domain.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18 and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO:17; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the CAR comprises (i) an antigen-binding domain comprising the amino acid sequence set forth in SEQ ID NO: 16; (ii) the intracellular domain of CD3z and a 4-1BB co-stimulatory domain; (iii) a transmembrane domain comprising the transmembrane domain of CD28; and (iv) an IgG hinge domain.

In some aspects, the CAR comprises (i) an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain. In some aspects, the CAR comprises (i) an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 17; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 21. In some aspects, the CAR comprises (i) an antigen-binding domain comprising a VH comprising the amino acid sequence set forth in SEQ ID NO: 7 and a VL comprising the amino acid sequence set forth in SEQ ID NO: 8; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain.

In some aspects, the CAR comprises (i) an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; (ii) the intracellular domain of CD3z comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 18, a CD28 co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 19, and a 4-1BB co-stimulatory domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO:17; (iii) a transmembrane domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 20; and (iv) an IgG hinge domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO:21. In some aspects, the CAR comprises (i) an antigen-binding domain comprising the amino acid sequence set forth in SEQ ID NO: 16; (ii) the intracellular domain of CD3z, a CD28 co-stimulatory domain, and a 4-1BB co-stimulatory domain; (iii) a transmembrane domain comprising the transmembrane domain of CD28; and (iv) an IgG hinge domain.

In some aspects, the nucleotide sequence encoding the CAR has at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 22. In some aspects, the nucleotide sequence encoding the CAR comprises the nucleotide sequence set forth in SEQ ID NO: 22.

### I.E. Armoring Molecule

Disclosed herein are polynucleotides comprising (a) a nucleotide sequence encoding a CAR, wherein the CAR comprises an antigen-binding domain that binds an epitope on human STEAP2, and (b) a nucleotide sequence encoding an armoring molecule. One approach to making CAR-T cells that are more resistant to tumor-associated immunosuppression is called “armoring.” Armoring is the molecular manipulation of a CAR-T cell to express one or more “armoring molecules” that can counter immunosuppression. For example, investigators reported modifying CAR-T cells to secrete PD-1-blocking single-chain variable fragments (scFv), which improved CAR-T cell anti-tumor activity in mouse models of PD-L1+ hematologic and solid tumors (Rafiq, S., Yeku, O., Jackson, H. et al. Targeted delivery of a PD-1-blocking scFv by CAR-T cells enhances anti-tumor efficacy in vivo. Nat Biotechnol 36, 847-856 (2018)). Others studies have demonstrated the effectiveness of armoring T cells with a dominant-negative TGF-β receptor type 2 (TGFβRIIDN) armoring molecule to neutralize the suppressive effects of TGF-β on T cells (Bollard et al., Tumor-Specific T-Cells Engineered to Overcome Tumor Immune Evasion Induce Clinical Responses in Patients With Relapsed Hodgkin Lymphoma, J Clin Oncol 36(11):121-1139 (2018)). Currently, at least one clinical study is investigating the effectiveness of armoring anti-PSMA-CAR-T cells with a TGFβRIIDN armoring molecule for treating castrate-resistant prostate cancer (NCT03089203).

In some aspects, the armoring molecule comprises a dominant-negative TGF-β receptor type 2 (TGFβRIIDN). In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the armoring molecule comprises the amino acid sequence set forth in SEQ ID NO: 10.

In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 105. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising the amino acid sequence set forth in SEQ ID NO: 10.

In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising the amino acid sequence set forth in SEQ ID NO: 10.

In some aspects, the armoring molecule comprises a dominant-negative TGF-β receptor type 2 (TGFβRIIDN). In some aspects, the armoring molecule comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the armoring molecule comprises the amino acid sequence set forth in SEQ ID NO: 10.

In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising the amino acid sequence set forth in SEQ ID NO: 10.

In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the polynucleotide comprises (a) a nucleotide sequence encoding a CAR, wherein the CAR comprises an antigen-binding domain comprising a V_{H} comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising the amino acid sequence set forth in SEQ ID NO: 10.

In some aspects, the polynucleotide comprises (a) a nucleotide sequence encoding a CAR, wherein the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; and (b) a nucleotide sequence encoding a TGFβRIIDN. In some aspects, the polynucleotide comprises (a) a nucleotide sequence encoding a CAR, wherein the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the polynucleotide comprises (a) a nucleotide sequence encoding a CAR, wherein the CAR comprises an antigen-binding domain comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16; and (b) an armoring molecule comprising the amino acid sequence set forth in SEQ ID NO: 10.

In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 12; and (b) a nucleotide sequence encoding a TGFβRIIDN. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 12; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the polynucleotide comprises (a) a nucleotide sequence encoding an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 12; and (b) an armoring molecule comprising the amino acid sequence set forth in SEQ ID NO: 10.

In some aspects, the polynucleotide comprises (a) a nucleotide sequence encoding a CAR comprising the nucleotide sequence set forth in SEQ ID NO: 22; and (b) a nucleotide sequence encoding an armoring molecule comprising the nucleotide sequence set forth in SEQ ID NO: 9.

In some aspects, the polynucleotide comprises (a) a nucleotide sequence encoding a CAR comprising the nucleotide sequence set forth in SEQ ID NO: 11; and (b) a nucleotide sequence encoding an armoring molecule comprising the nucleotide sequence set forth in SEQ ID NO: 9.

In some aspects, the nucleotide encoding the CAR and the nucleotide encoding the armoring molecule are linked by a third nucleotide sequence, wherein the third nucleotide sequence encodes a cleavable peptide linker. In some aspects, the cleavable peptide linker comprises a T2A peptide. In some aspects, the cleavable peptide linker comprises SEQ ID NO: 13.

In some aspects, the polynucleotide comprises a nucleotide sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the nucleotide sequence set forth in SEQ ID NO: 14. In some aspects, the polynucleotide comprises the nucleotide sequence set forth in SEQ ID NO: 14.

### III. Anti-STEAP2 Antibodies of the Present Disclosure

Some aspects of the present disclosure are directed to antibodies or antigen-binding portions thereof that specifically binds human STEAP2. In some aspects, the antibody or antigen-binding portion thereof comprises a variable heavy chain region (VH) and a variable light chain region (VL), wherein the VH comprises a VH complementarity determining region (CDR) 1, a VH-CDR2, a VH-CDR3; and wherein the VL comprises a VL-CDR1, a VL-CDR2, and VL-CDR3. In some aspects, the antibody or antigen-binding portion thereof comprises a VH-CDR3 comprising an amino acid sequence selected from SEQ ID NOs: 6. In some aspects, the antibody or antigen-binding portion thereof comprises a VH-CDR2 comprising an amino acid sequence selected from SEQ ID NOs: 5. In some aspects, the antibody or antigen-binding portion thereof comprises a VH-CDR1 comprising an amino acid sequence selected from SEQ ID NOs: 4.

In some aspects, the antibody or antigen-binding portion thereof comprises a VL-CDR3 comprising an amino acid sequence selected from SEQ ID NOs: 3. In some aspects, the antibody or antigen-binding portion thereof comprises a VL-CDR2 comprising an amino acid sequence selected from SEQ ID NOs: 2. In some aspects, the antibody or antigen-binding portion thereof comprises a VL-CDR1 comprising an amino acid sequence selected from SEQ ID NOs: 1.

In some aspects, the antibody or antigen-binding portion thereof comprises a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 7. In some aspects, the antibody or antigen-binding portion thereof comprises a VH comprising an amino acid sequence selected from SEQ ID NOs: 7.

In some aspects, the antibody or antigen-binding portion thereof comprises a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to an amino acid sequence selected from SEQ ID NOs: 8. In some aspects, the antibody or antigen-binding portion thereof comprises a VL comprising an amino acid sequence selected from SEQ ID NOs: 8.

In some aspects, the antibody or antigen-binding portion thereof comprises a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7, and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the antibody or antigen-binding portion thereof comprises a VH comprising the amino acid sequence set forth in SEQ ID NO: 7, and a VL comprising the amino acid sequence set forth in SEQ ID NO: 8.

In some aspects, the antibody or antigen-binding portion thereof comprises an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 16. In some aspects, the antibody or antigen-binding portion thereof comprises the amino acid sequence set forth in SEQ ID NO: 16.

In some aspects, the antibody or antigen-binding portion thereof cross competes for binding to human STEAP2 with an antibody or antigen-binding portion thereof disclosed herein. In some aspects, the antibody or antigen-binding portion thereof binds the same epitope on human STEAP2 as an antibody or antigen-binding portion thereof disclosed herein. In some aspects, the antibody or antigen-binding portion thereof binds on overlapping epitope on human STEAP2 as an antibody or antigen-binding portion thereof disclosed herein.

### IV. Androgen Receptor Antagonists of the Present Disclosure

Androgen receptor is a member of the nuclear hormone receptor family activated by androgens, such as dihydrotestosterone (DHT). Androgen receptor is a prime therapeutic target for treating prostate cancer. Several compounds have been developed as chemotherapy for prostate cancer.

Androgen receptor antagonists (antiandrogens) are drugs used to treat hormonal-based syndromes and prostate cancer. Current drugs for prostate cancer include flutamide, bicalutamide, nilutamide, enzalutamide and ARN-509. Each of these inhibitors binds to the hormone-binding pocket (HBP) of the androgen receptor. This is the same site that the natural physiological steroids testosterone (TES) and dihydrotestosterone (DHT) bind. The drugs work by competing with the natural hormones for binding to the pocket and, as a result, lessening activation of the receptor.

Castration-resistant prostate cancer has been shown to be sensitive, but not resistant, to sustained manipulation of the androgen/AR axis. The androgen axis can be manipulated using anti-androgens (nilutamide, enzalutamide), androgen synthesis inhibitors (ketonazole, abiraterone acetate), corticosteroids (dexamethasone, prednisone) or estrogen treatment. Following the emergence of castration-refractory disease, taxane-based chemotherapy has been shown to be therapeutically efficacious and prolong survival. Patients progressing on docetaxel have been shown to benefit from abiraterone acetate, a selective cytochrome P450 17A1 inhibitor which requires co-administration with glucocorticoids to curtail side effects. Enzalutamide (MDV-3100) is a novel AR antagonist that blocks AR signaling more effectively than currently available AR antagonists (Tran et al., Science 2009; 324(5928): 787-790.) and has shown impressive antitumor activity and a similar impact on overall survival as abiraterone.

Some aspects of the present disclosure are directed to a method for inhibiting the growth of a tumor cell comprising contacting said tumor cell with: a T cell comprising (i) a polynucleotide encoding a chimeric antigen receptor (CAR) that binds an epitope on human six transmembrane epithelial antigen of prostate-2 (STEAP2); and at least one androgen receptor antagonist, in an amount effective to inhibit tumor cell growth. In some aspects, the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

Some aspects of the present disclosure are directed to a method for inhibiting the growth of a tumor cell comprising contacting said tumor cell with: a T cell comprising (i) a polynucleotide encoding a chimeric antigen receptor (CAR) that binds an epitope on human six transmembrane epithelial antigen of prostate-2 (STEAP2); and at least one androgen receptor antagonist, in an amount effective to inhibit tumor cell growth; wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

Some aspects of the present disclosure are direct to a method for treating a cancer comprising tumor cells in a subject comprising administering to said subject in need thereof a therapeutically effective amount of: a T cell comprising (i) a polynucleotide encoding a chimeric antigen receptor (CAR) that binds an epitope on human six transmembrane epithelial antigen of prostate-2 (STEAP2); and at least one androgen receptor antagonist. In some aspects, the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

Some aspects of the present disclosure are direct to a method for treating a cancer comprising tumor cells in a subject comprising administering to said subject in need thereof a therapeutically effective amount of: a T cell comprising (i) a polynucleotide encoding a chimeric antigen receptor (CAR) that binds an epitope on human six transmembrane epithelial antigen of prostate-2 (STEAP2); and at least one androgen receptor antagonist; wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

In aspects, the androgen receptor is a mutant androgen receptor. In aspects, the mutant androgen receptor is associated with a disease that is not associated with wildtype androgen receptor. In aspects, the androgen receptor includes at least one amino acid mutation (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 mutations) compared to the sequence above. In aspects, the mutant androgen receptor is a splice variant. In aspects, the mutant androgen receptor is lacking a portion of the ligand binding domain. In aspects, the mutant androgen receptor is active in the absence of bound ligand. In aspects, the mutant androgen receptor is lacking the ligand binding domain. In aspects, the splice variant androgen receptor is AR variant 1 (e.g., GI:21322252). In aspects, the splice variant androgen receptor is AR variant 2 (AR45) (e.g., GI:21713434). In aspects, the splice variant androgen receptor is AR variant 3 (AR-V7) (e.g., GI:224181614). In aspects, the splice variant androgen receptor is AR variant 4 (AR-V1) (e.g., GI:224181616). In aspects, the splice variant androgen receptor is AR variant 5 (AR-V4) (e.g., GI:224181620). In aspects, the splice variant androgen receptor is AR variant 6 (AR-V3) (e.g., GI:224181622). In aspects, the splice variant androgen receptor is AR v567es (e.g., GI:270358642).

In aspects, the androgen receptor antagonist is an antagonist of a nuclear receptor. In aspects, the androgen receptor antagonist is an antagonist of a human androgen receptor. In aspects, the androgen receptor antagonist is an antagonist of wildtype human androgen receptor. In aspects, the androgen receptor antagonist is an antagonist of a mutant human androgen receptor. In aspects, the androgen receptor antagonist is an antagonist of a drug-resistant human androgen receptor. In aspects, the androgen receptor antagonist is an antagonist of a casodex-resistant human androgen receptor. In aspects, the androgen receptor antagonist is an antagonist of a Flutamide-resistant human androgen receptor. In aspects, the androgen receptor antagonist is an antagonist of an MDV3100-resistant human androgen receptor. In aspects, the androgen receptor antagonist is an antagonist of an ARN-509-resistant human androgen receptor. In aspects, the androgen receptor antagonist is an antagonist of non-ligand activated androgen receptor. In aspects, the androgen receptor antagonist is an antagonist of N-terminal activated non-ligand activated androgen receptor. In aspects, the androgen receptor antagonist is an antagonist of a non-ligand activated androgen receptor splice variant. In aspects, the androgen receptor antagonist is an antagonist of a non-ligand activated androgen receptor activated by HER2. In aspects, the androgen receptor antagonist is an antagonist of a non-ligand activated androgen receptor activated by IL-6.

In aspects, the androgen receptor antagonist does not inhibit ligand (e.g., DHT) binding to androgen receptor. In aspects, the androgen receptor antagonist does not bind the ligand binding domain of androgen receptor. In aspects, the androgen receptor antagonist binds the DNA binding domain of androgen receptor. In aspects, the androgen receptor antagonist does not increase the degradation of androgen receptor relative to the absence of the androgen receptor antagonist. In aspects, the androgen receptor antagonist does not reduce the nuclear localization of androgen receptor relative to the absence of the androgen receptor antagonist. In aspects, the androgen receptor antagonist does not prevent androgen receptor binding to DNA. In aspects, the androgen receptor antagonist does not reduce the binding of androgen receptor to DNA. In aspects, the androgen receptor antagonist prevents the recruitment of RNA pol II to DNA. In aspects, the androgen receptor antagonist prevents the binding of RNA pol II to the transcription complex including androgen receptor bound to the androgen receptor antagonist. In aspects, the androgen receptor antagonist prevents initiation of transcription by androgen receptor. In aspects, the androgen receptor antagonist inhibits (e.g., compared to control) the recruitment of RNA pol II to DNA. In aspects, the androgen receptor antagonist inhibits (e.g., compared to control) the binding of RNA pol II to the transcription complex including androgen receptor bound to the androgen receptor antagonist. In aspects, the androgen receptor antagonist inhibits (e.g., compared to control) initiation of transcription by androgen receptor. In aspects, the androgen receptor antagonist reduces transcription induced by androgen receptor relative to control (e.g., absence of androgen receptor antagonist). In aspects, the androgen receptor antagonist reduces co-activator binding to androgen receptor. In aspects, the androgen receptor antagonist binds androgen receptor and DNA. In aspects, the androgen receptor antagonist binds androgen receptor while bound to DNA. In aspects, the androgen receptor antagonist binds the minor groove of DNA. In aspects, the androgen receptor antagonist contacts one or more of Lys609, Asn610, Pro612, Phe582, Ala586, Tyr593, and/or Arg615. In aspects, the androgen receptor antagonist contacts one or more of Lys609, Asn610, and Pro612. In aspects, the androgen receptor antagonist contacts the minor groove of DNA. In aspects, the androgen receptor antagonist contacts one or more amino acids corresponding to Lys609, Asn610, Pro612, Phe582, Ala586, Tyr593, and/or Arg615 of human androgen receptor. In aspects, the androgen receptor antagonist contacts one or more amino acids corresponding to Lys609, Asn610, and/or Pro612 of human androgen receptor. In aspects, the androgen receptor antagonist contacts amino acids corresponding to Lys609 and Pro612 of human androgen receptor. In aspects, the androgen receptor antagonist contacts amino acids corresponding to Lys609 and Pro612 but not Asn610 of human androgen receptor. In aspects, the androgen receptor antagonist contacts amino acids corresponding to Lys609 or Pro612 but not Asn610 of human androgen receptor. In aspects, the androgen receptor antagonist contacts amino acids corresponding to Lys609 or Pro612 of human androgen receptor. In aspects, the androgen receptor antagonist contacts amino acids Lys609 or Pro612 of human androgen receptor. In aspects, the androgen receptor antagonist contacts amino acids Lys609 and Pro612 of human androgen receptor. In aspects, the androgen receptor antagonist contacts amino acids Lys609 and Pro612 but not Asn610 of human androgen receptor. In aspects, the androgen receptor antagonist contacts amino acids Lys609 or Pro612 and not Asn610 of human androgen receptor.

In some aspects, the androgen receptor antagonist inhibits (e.g. compared to control) androgen receptor activity in prostate and/or bone to a greater degree than other tissues.

In some aspects, the androgen receptor antagonist is soluble in an aqueous solution. In some aspects, the androgen receptor antagonist is soluble in a dextrin (e.g., hydroxypropyl beta and gamma).

In some aspects, the androgen receptor antagonist is flutamide, nilutamide, enzalutamide, bicalutamide, ketonazole, abiraterone, abiraterone acetate, orteronel, finasteride, dutasteride, bexlosteride, izonsteride, turosteride, episteride, dexamethasone, prednisone, leuprolide, goserelin, triptorelin, histrelin, estrogen, or a combination thereof.

In some aspects, the androgen receptor antagonist is enzalutamide, apalutamide, darolutamide, abiraterone, bicalutamide, nilutamide, flutamide, proxalutamide, or a combination thereof.

In some aspects, the androgen receptor antagonist is enzalutamide (Tran et al., Science 2009, 324(5928): 787-790.) Enzalutamide can be obtained from, for example, Medivation or Astellas under the name Xtandi®. The structure of enzalutamide is shown below (Xtandi® product label; 08/2012 revision):

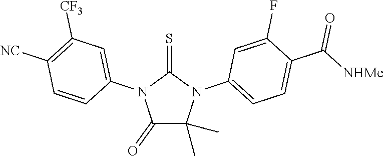 [SMILES: CNC(=O)c1ccc(N2C(=S)N(c3ccc(C#N)c(C(F)(F)F)c3)C(=O)C2(C)C)cc1F]

In some aspects, the androgen receptor antagonist is abiraterone, for example in the form of abiraterone acetate (Agarwal et al., Future Oncology 2010, 6(5): 665-679). Abiraterone can be obtained from, for example, Janssen Biotech, Inc. The structure of abiraterone acetate is shown below (ZYTIGA® product label; 03/2015 revision):

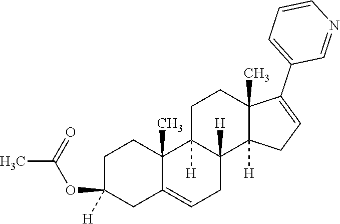

In some aspects, the androgen receptor antagonist is administered orally.

In some aspects, administration of the androgen receptor antagonist does not inhibit T cell viability or T cell killing capacity (e.g, viability or killing capacity of an anti-STEAP2 CAR T cell).

In some aspects, administration of the androgen receptor antagonist does not inhibit interferon-gamma (IFNγ) release by a CAR T cell (e.g., an anti-STEAP2 CAR T cell) in tumor cells.

In some aspects, administration of the androgen receptor antagonist does not reduce CAR expression on the T cell (e.g., an anti-STEAP2 CAR on the T cell).

### V. Cells of the Disclosure

Some aspects of the present disclosure are directed to cells comprising a polynucleotide or a polypeptide disclosed herein. Some aspects of the present disclosure are directed to a cell comprising (i) a polynucleotide encoding a chimeric antigen receptor (CAR) that binds human STEAP2. In some aspects, the cell further comprises (ii) a polynucleotide encoding an armoring molecule. In some aspects, the cell is an immune cell. In some aspects, the cell is selected from the group consisting of a T cell, a Natural Killer (NK) cell, a cytotoxic T lymphocyte (CTL), a regulatory T cell, a tumor infiltrating lymphocyte, and any combination thereof. In some aspects, the cell is a mammalian cell. In some aspects, the cell is a human cell.

The cell of the present disclosure can be obtained through any source. For example, T cells can be differentiated in vitro from a hematopoietic stem cell population, or T cells can be obtained from a subject. T cells can be obtained from, e.g., peripheral blood mononuclear cells, bone marrow, lymph node tissue, cord blood, thymus tissue, tissue from a site of infection, ascites, pleural effusion, spleen tissue, and tumors. In addition, the T cells can be derived from one or more T cell lines available in the art. T cells can also be obtained from a unit of blood collected from a subject using any number of techniques known to the skilled artisan, such as FICOLL™ separation and/or apheresis. In certain aspects, the cells collected by apheresis are washed to remove the plasma fraction, and placed in an appropriate buffer or media for subsequent processing. In some aspects, the cells are washed with PBS. As will be appreciated, a washing step can be used, such as by using a semiautomated flowthrough centrifuge, e.g., the COBE™ 2991 cell processor, the Baxter CYTOMATE™, or the like. In some aspects, the washed cells are resuspended in one or more biocompatible buffers, or other saline solution with or without buffer. In certain aspects, the undesired components of the apheresis sample are removed. Additional methods of isolating T cells for a T cell therapy are disclosed in U.S. Patent Publication No. 2013/0287748, which is herein incorporated by references in its entirety.

In certain aspects, T cells are isolated from PBMCs by lysing the red blood cells and depleting the monocytes, e.g., by using centrifugation through a PERCOLL™ gradient. In some aspects, a specific subpopulation of T cells, such as CD28+, CD4+, CD8+, CD45RA+, and CD45RO+ T cells is further isolated by positive or negative selection techniques known in the art. For example, enrichment of a T cell population by negative selection can be accomplished with a combination of antibodies directed to surface markers unique to the negatively selected cells. In some aspects, cell sorting and/or selection via negative magnetic immunoadherence or flow cytometry that uses a cocktail of monoclonal antibodies directed to cell surface markers present on the cells negatively selected can be used. For example, to enrich for CD4+ cells by negative selection, a monoclonal antibody cocktail typically includes antibodies to CD14, CD20, CD11b, CD16, HLA-DR, and CD8. In certain aspects, flow cytometry and cell sorting are used to isolate cell populations of interest for use in the present disclosure.

In some aspects, PBMCs are used directly for genetic modification with the immune cells (such as CARs) using methods as described herein. In certain aspects, after isolating the PBMCs, T lymphocytes are further isolated, and both cytotoxic and helper T lymphocytes are sorted into naive, memory, and effector T cell subpopulations either before or after genetic modification and/or expansion.

In some aspects, CD8+ cells are further sorted into naive, central memory, and effector cells by identifying cell surface antigens that are associated with each of these types of CD8+ cells. In some aspects, the expression of phenotypic markers of central memory T cells includes CD45RO, CD62L, CCR7, CD28, CD3, and CD127 and are negative for granzyme B. In some aspects, central memory T cells are CD45RO+, CD62L+, CD8+ T cells. In some aspects, effector T cells are negative for CD62L, CCR7, CD28, and CD127 and positive for granzyme B and perforin. In certain aspects, CD4+ T cells are further sorted into subpopulations. For example, CD4+T helper cells can be sorted into naive, central memory, and effector cells by identifying cell populations that have cell surface antigens.

In some aspects, the immune cells, e.g., T cells, are genetically modified following isolation using known methods, or the immune cells are activated and expanded (or differentiated in the case of progenitors) in vitro prior to being genetically modified. In another aspect, the immune cells, e.g., T cells, are genetically modified with the CARs described herein (e.g., transduced with a viral vector comprising one or more nucleotide sequences encoding a CAR) and then are activated and/or expanded in vitro. Methods for activating and expanding T cells are known in the art and are described, e.g., in U.S. Pat. Nos. 6,905,874; 6,867,041; and 6,797,514; and PCT Publication No. WO 2012/079000, the contents of which are hereby incorporated by reference in their entirety. Generally, such methods include contacting PBMC or isolated T cells with a stimulatory agent and costimulatory agent, such as anti-CD3 and anti-CD28 antibodies, generally attached to a bead or other surface, in a culture medium with appropriate cytokines, such as IL-2. Anti-CD3 and anti-CD28 antibodies attached to the same bead serve as a “surrogate” antigen presenting cell (APC). One example is The Dynabeads® system, a CD3/CD28 activator/stimulator system for physiological activation of human T cells. In other aspects, the T cells are activated and stimulated to proliferate with feeder cells and appropriate antibodies and cytokines using methods such as those described in U.S. Pat. Nos. 6,040,177 and 5,827,642 and PCT Publication No. WO 2012/129514, the contents of which are hereby incorporated by reference in their entirety.

In certain aspects, the T cells are obtained from a donor subject. In some aspects, the donor subject is human patient afflicted with a cancer or a tumor. In other aspects, the donor subject is a human patient not afflicted with a cancer or a tumor.

In some aspects, the cell comprises a polynucleotide comprising a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the cell comprises a polynucleotide comprising a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8.

In some aspects, the cell comprises a polynucleotide comprising a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6. In some aspects, the polynucleotide comprises a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the cell comprises a polynucleotide comprising a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8. In some aspects, the polynucleotide comprises a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8.

In some aspects, the cell comprises a polynucleotide comprising (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising the amino acid sequence set forth in SEQ ID NO: 10.

In some aspects, the cell comprises a polynucleotide comprising (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 10. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising the amino acid sequence set forth in SEQ ID NO: 10.

In some aspects, the cell comprises a polynucleotide comprising (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) a nucleotide sequence encoding a TGFβRIIDN. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising a nucleic acid sequence having at least about 70%, at least about 75%, at least about 80%, at least about 85%, at least about 90%, at least about 95%, at least about 96%, at least about 97%, at least about 98%, or at least about 99% sequence identity to the nucleic acid sequence set forth in SEQ ID NO: 9. In some aspects, the polynucleotide comprises (a) a nucleotide sequence encoding a CAR, wherein the CAR comprises an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6; and (b) an armoring molecule comprising the nucleic acid sequence set forth in SEQ ID NO: 9.

In some aspects, the cell comprises a polynucleotide comprising (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) a nucleotide sequence encoding a TGFβRIIDN. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising nucleic acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 9. In some aspects, the polynucleotide comprises (a) a nucleotide sequence encoding a CAR, wherein the CAR comprises an antigen-binding domain comprising a VH comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 7 and a VL comprising an amino acid sequence having at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 8; and (b) an armoring molecule comprising the nucleic acid sequence set forth in SEQ ID NO: 9.

In some aspects, the cell comprises a polynucleotide comprising (a) a nucleotide sequence encoding a CAR comprising the nucleotide sequence set forth in SEQ ID NO: 22; and (b) a nucleotide sequence encoding an armoring molecule comprising the nucleotide sequence set forth in SEQ ID NO: 9.

In some aspects, the cell comprises a polynucleotide comprising a nucleotide sequence encoding a polypeptide comprising an amino acid sequence having at least about 60%, at least about 65%, at least about 70%, at least about 75%, at least about 80%, at least about 85%, at least about 90%, at least about 95%, at least about 96%, at least about 97%, at least about 98%, or at least about 99% sequence identity to the amino acid sequence set forth in SEQ ID NO: 15. In some aspects, the polypeptide comprises the amino acid sequence set forth in SEQ ID NO: 15.

In some aspects, the cell comprises a CAR comprising an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6.

In some aspects, the cell comprises a polypeptide that comprises (i) a CAR comprising an antigen-binding domain comprising a VL-CDR1 comprising the amino acid sequence set forth in SEQ ID NO: 1, a VL-CDR2 comprising the amino acid sequence set forth in SEQ ID NO: 2, a VL-CDR3 comprising the amino acid sequence set forth in SEQ ID NO: 3, a VH-CDR1 comprising the amino acid sequence set forth in SEQ ID NO: 4, a VH-CDR2 comprising the amino acid sequence set forth in SEQ ID NO: 5, a VH-CDR3 comprising the amino acid sequence set forth in SEQ ID NO: 6 and (ii) an amino acid sequence set forth in SEQ ID NO: 10.

In some aspects, the cell comprises a CAR comprising an amino acid set forth in SEQ ID NO: 12 and an armoring molecule comprising an amino acid set forth in SEQ ID NO: 10.

### VI. Vectors, Host Cells, and Pharmaceutical Compositions of the Disclosure

In some aspects, the polynucleotide of the present disclosure is present in a vector. As such, provided herein are vectors comprising a polynucleotide of the present disclosure. In some aspects, the present disclosure is directed to a vector or a set of vectors comprising a polynucleotide encoding a CAR, as described herein. In other aspects, the present disclosure is directed to a vector or a set of vectors comprising a polynucleotide encoding an antibody or an antigen binding molecule thereof that specifically binds to STEAP2, as disclosed herein.

In some aspects, the set of vectors comprises a first vector and a second vector, wherein the first vector comprises a nucleic acid sequence encoding a CAR disclosed herein, and the second vector comprises a nucleic acid sequence encoding an armoring molecule disclosed herein.

Any vector known in the art can be suitable for the present disclosure. In some aspects, the vector is a viral vector. In some aspects, the vector is a retroviral vector, a DNA vector, a murine leukemia virus vector, an SFG vector, a plasmid, a RNA vector, an adenoviral vector, a baculoviral vector, an Epstein Barr viral vector, a papovaviral vector, a vaccinia viral vector, a herpes simplex viral vector, an adenovirus associated vector (AAV), a lentiviral vector, or any combination thereof.

In other aspects, provided herein are host cells comprising a polynucleotide or a vector of the present disclosure. In some aspects, the present disclosure is directed to host cells, e.g., in vitro cells, comprising a polynucleotide encoding a CAR or a TCR, as described herein. In some aspects, the present disclosure is directed to host cells, e.g., in vitro cells, comprising a polynucleotide encoding an antibody or an antigen binding molecule thereof that specifically binds to STEAP2, as disclosed herein. In other aspects, the present disclosure is directed to in vitro cells comprising a polypeptide encoded by a polynucleotide encoding a CAR-That specifically binds to STEAP2. In other aspects, the present disclosure is directed to cells, in vitro cells, comprising a polypeptide encoded by a polynucleotide encoding an antibody or an antigen binding molecule thereof that specifically binds to STEAP2, as disclosed herein.

Any cell may be used as a host cell for the polynucleotides, the vectors, or the polypeptides of the present disclosure. In some aspects, the cell can be a prokaryotic cell, fungal cell, yeast cell, or higher eukaryotic cells such as a mammalian cell. Suitable prokaryotic cells include, without limitation, eubacteria, such as Gram-negative or Gram-positive organisms, for example, Enterobactehaceae such as Escherichia, e.g., E. coli; Enterobacter; Erwinia; Klebsiella; Proteus; Salmonella, e.g., Salmonella typhimurium; Serratia, e.g., Serratia marcescans, and Shigella; Bacilli such as B. subtilis and B. licheniformis; Pseudomonas such as P. aeruginosa; and Streptomyces. In some aspects, the cell is a human cell.

Other aspects of the present disclosure are directed to compositions comprising a polynucleotide described herein, a vector described herein, a polypeptide described herein, or cell described herein. In some aspects, the composition comprises a pharmaceutically acceptable carrier, diluent, solubilizer, emulsifier, preservative and/or adjuvant. In some aspects, the composition comprises an excipient. In one aspect, the composition comprises a polynucleotide encoding a CAR, wherein the CAR comprises an antigen binding molecule that specifically binds to STEAP2. In another aspect, the composition comprises a CAR encoded by a polynucleotide of the present disclosure, wherein the CAR comprises an antigen binding molecule that specifically binds to STEAP2. In another aspect, the composition comprises a T cell comprising a polynucleotide encoding a CAR, wherein the CAR comprises an antigen binding molecule that specifically binds to STEAP2. In another aspect, the composition comprises an antibody or an antigen binding molecule thereof that specifically binds STEAP2, as described herein. In another aspect, the composition comprises a cell (e.g., a T cell, e.g., a CAR-T cell) comprising a polynucleotide encoding CAR comprising an antigen binding domain that specifically binds STEAP2, as disclosed herein. In another aspect, the composition further comprises at least one androgen receptor antagonist.

The disclosure also provides pharmaceutical compositions comprising an anti-STEAP2 CAR T cell and a pharmaceutically acceptable carrier. In some aspects, the pharmaceutical composition further comprises at least one androgen receptor antagonist. The pharmaceutical compositions comprising an anti-STEAP2 CAR T cell are for use in, but not limited to, diagnosing, detecting, or monitoring a disorder, in preventing, treating, managing, or ameliorating of a disorder or one or more symptoms thereof, and/or in research. In an aspect, provided herein is a pharmaceutical composition comprising an anti-STEAP2 CAR T cell as described herein and a pharmaceutically acceptable carrier. In some aspects, provided herein is a pharmaceutical composition comprising at least one androgen receptor antagonist as described herein and a pharmaceuticall acceptable carrier.

The disclosure also provides pharmaceutical compositions comprising an anti-STEAP2 CAR T cell, at least one androgen receptor antagonist, and a pharmaceutically acceptable carrier. The pharmaceutical compositions comprising an anti-STEAP2 CAR T cell and at least one androgen receptor antagonist are for use in, but not limited to, diagnosing, detecting, or monitoring a disorder, in preventing, treating, managing, or ameliorating of a disorder or one or more symptoms thereof, and/or in research. In an aspect, provided herein is a pharmaceutical composition comprising an anti-STEAP2 CAR T cell and at least one androgen receptor antagonist as described herein and a pharmaceutically acceptable carrier.

In other aspects, the composition is formulated for parenteral delivery, for inhalation, or for delivery through the digestive tract, such as orally. The preparation of such pharmaceutically acceptable compositions is within the ability of one skilled in the art. In certain aspects, buffers are used to maintain the composition at physiological pH or at a slightly lower pH, typically within a pH range of from about 5 to about 8. In certain aspects, when parenteral administration is contemplated, the composition is in the form of a pyrogen-free, parenterally acceptable aqueous solution comprising a desired antigen binding molecule to BCMA, with or without additional therapeutic agents, in a pharmaceutically acceptable vehicle. In certain aspects, the vehicle for parenteral injection is sterile distilled water in which an antigen binding molecule to BCMA, with or without at least one additional therapeutic agent, is formulated as a sterile, isotonic solution, properly preserved. In certain aspects, the preparation involves the formulation of the desired molecule with polymeric compounds (such as polylactic acid or polyglycolic acid), beads or liposomes, that provide for the controlled or sustained release of the product, which are then be delivered via a depot injection. In certain aspects, implantable drug delivery devices are used to introduce the desired molecule.

A pharmaceutical composition is formulated to be compatible with its intended route of administration. Examples of routes of administration include, but are not limited to, parenteral, e.g., intravenous, intradermal, subcutaneous, intranasal (e.g., inhalation), transdermal (e.g., topical), transmucosal, and rectal administration. In a specific aspect, the composition is formulated in accordance with routine procedures as a pharmaceutical composition adapted for intravenous, subcutaneous, intramuscular, intranasal, or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

In various aspects, kits for use in the laboratory and therapeutic applications described herein are within the scope of the present disclosure. Such kits may comprise a carrier, package, or container that is compartmentalized to receive one or more containers such as vials, tubes, and the like, each of the container(s) comprising one of the separate elements to be used in a method disclosed herein, along with a label or insert comprising instructions for use, such as a use described herein. Kits may comprise a container comprising a drug moiety. The present disclosure also provides one or more of the anti-STEAP2 CAR T cell and at least one androgen receptor antagonist, or pharmaceutical compositions thereof, packaged in a hermetically sealed container, such as an ampoule or sachette, indicating the quantity of the agent.

Kits may comprise the container described above, and one or more other containers associated therewith that comprise materials desirable from a commercial and user standpoint, including buffers, diluents, filters, needles, syringes; carrier, package, container, vial and/or tube labels listing contents and/or instructions for use, and package inserts with instructions for use.

A label may be present on or with the container to indicate that the composition is used for a specific therapy or non-therapeutic application, such as a prognostic, prophylactic, diagnostic, or laboratory application. A label may also indicate directions for either in vivo or in vitro use, such as those described herein. Directions and or other information may also be included on an insert(s) or label(s), which is included with or on the kit. The label may be on or associated with the container. A label may be on a container when letters, numbers, or other characters forming the label are molded or etched into the container itself. A label may be associated with a container when it is present within a receptacle or carrier that also holds the container, e.g., as a package insert. The label may indicate that the composition is used for diagnosing or treating a condition, such as a cancer a described herein.

TABLE 1
Sequences.

| SEQ | | |
|---|---|---|
| ID | Description | Sequences |
| 1 | STEAP2 | RASQSVNSNLA |
| | VL CDR1 | |
| | (40A3) | |
| 2 | STEAP2 | GASTRAT |
| | VL CDR2 | |
| 3 | STEAP2 | QQYNNWPFT |
| | VL CDR3 | |
| 4 | STEAP2 | RNSAVWN |
| | VH CDR1 | |
| 5 | STEAP2 | RTYYRSKWYNDYAVSVKS |
| | VH CDR2 | |
| 6 | STEAP2 | GLLQNNFYYYMDV |
| | VH CDR3 | |
| 7 | STEAP2 | QVQLQQSGPGLVKPSQTLSLTCAISGDSVSRNSAVWNWIRQS |
| | VH | PSRGLEWLGRTYYRSKWYNDYAVSVKSRITINPDTSKNQFSL |
| | | QVNSVTPEDTAVYYCARGLLQNNFYYYMDVWGKGTTVTVSS |
| 8 | STEAP2 | EIVMTQSPATLSVSPGERATLSCRASQSVNSNLAWYQQKPGQ |
| | VL | APRLLIYGASTRATGIPARFSGSGSGTEFTLTISSLQSEDFA |
| | | VYYCQQYNNWPFTFGPGTKVDIK |
| 9 | Dominant- | ATGGGACGCGGGCTGCTTCGAGGACTCTGGCCACTTCATATC |
| | negative | GTGTTGTGGACTCGCATCGCTTCAACCATTCCGCCGCACGTG |
| | TGF-β | CAGAAGTCCGTGAACAATGACATGATCGTGACCGACAACAAC |
| | receptor | GGTGCAGTGAAGTTCCCACAGCTGTGCAAGTTCTGCGATGTC |
| | type 2 | AGATTCAGCACTTGCGACAACCAGAAGTCCTGCATGTCAAAC |
| | nucleic | TGCTCCATCACCTCCATCTGCGAGAAGCCTCAAGAGGTCTGC |
| | acid | GTGGCCGTGTGGCGGAAGAACGACGAGAACATCACCCTGGAA |
| | sequence | ACCGTGTGCCACGATCCGAAGCTGCCTTATCACGACTTCATT |
| | | CTGGAAGATGCCGCCTCGCCCAAGTGTATCATGAAAGAAAAG |
| | | AAAAAGCCCGGAGAAACGTTCTTCATGTGCTCGTGTAGCTCC |
| | | GACGAGTGCAACGACAACATTATCTTTAGCGAAGAGTACAAC |
| | | ACTTCCAACCCTGACCTCCTGCTCGTGATTTTTCAAGTCACC |
| | | GGCATTTCCCTGCTGCCCCCGCTGGGCGTGGCGATCTCGGTG |
| | | ATCATTATCTTCTACTGTTACCGGGTCAATAGGCAG |
| 10 | Dominant- | MGRGLLRGLWPLHIVLWTRIASTIPPHVQKSVNNDMIVTDNN |
| | negative | GAVKFPQLCKFCDVRESTCDNQKSCMSNCSITSICEKPQEVC |
| | TGF-β | VAVWRKNDENITLETVCHDPKLPYHDFILEDAASPKCIMKEK |
| | receptor | KKPGETFFMCSCSSDECNDNIIFSEEYNTSNPDLLLVIFQVT |
| | type 2 | GISLLPPLGVAISVIIIFYCYRVNRQ |
| | amino | |
| 11 | STEAP2 | ATGCTGCTCCTTGTCACATCACTGCTGCTCTGCGAACTGCCC |
| | | CACCCTGCATTCCTCCTGATCCCCGAGATTGTGATGACCCAG |
| | | AGCCCTGCAACTCTGAGCGTGTCACCCGGAGAAAGGGCCACT |
| | | CTGTCGTGTCGAGCATCGCAGTCCGTGAACTCCAATCTCGCC |
| | | TGGTACCAGCAGAAGCCTGGGCAGGCCCCGAGGCTGCTCATC |
| | | TACGGTGCCTCCACGAGAGCCACGGGAATTCCAGCGCGCTTT |
| | | AGCGGATCCGGCTCGGGAACCGAGTTCACCCTTACCATCTCA |
| | | TCGCTGCAATCCGAAGATTTCGCCGTGTATTACTGTCAACAG |
| | | TACAACAACTGGCCGTTCACCTTTGGCCCGGGAACTAAGGTC |
| | | GACATCAAGGGCGGCGGGGGCTCTGGGGGTGGCGGAAGCGGC |
| | | GGCGGCGGATCCGGTGGCGGCGGAAGCCAAGTGCAGCTGCAG |
| | | CAGTCCGGACCCGGACTCGTGAAGCCGTCCCAGACTCTGTCC |
| | | CTGACTTGCGCGATTTCCGGCGATTCCGTGTCCCGCAACTCC |
| | | GCTGTGTGGAACTGGATCCGGCAGTCGCCTTCGAGAGGACTG |
| | | GAGTGGCTGGGACGGACCTACTACCGCTCAAAATGGTATAAC |
| | | GACTATGCTGTGTCCGTCAAGAGCCGCATCACCATTAACCCC |
| | | GATACCTCCAAGAACCAGTTCAGTCTGCAAGTCAACAGCGTG |
| | | ACTCCTGAGGACACCGCCGTGTACTACTGCGCCCGGGGTCTG |
| | | CTGCAAAACAACTTCTACTACTACATGGACGTCTGGGGAAAG |
| | | GGAACTACTGTGACCGTGTCCTCCGGCTCCGAATCAAAATAC |
| | | GGTCCGCCATGCCCACCGTGCCCCTTCTGGGTGCTCGTGGTC |
| | | GTCGGAGGGGTTCTGGCCTGCTACTCCCTGCTGGTCACCGTG |
| | | GCGTTTATCATCTTCTGGGTGAAGCGGGGAAGGAAGAAGCTA |
| | | CTGTACATTTTCAAGCAGCCTTTCATGCGGCCTGTGCAGACC |
| | | ACCCAGGAAGAGGACGGCTGTTCCTGCCGGTTCCCCGAGGAA |
| | | GAGGAAGGGGGTTGCGAGCTGCGCGTGAAGTTCAGCAGGAGC |
| | | GCTGATGCCCCAGCGTACCAACAGGGGCAAAACCAGTTGTAC |
| | | AACGAACTGAACCTTGGTCGGCGCGAAGAGTACGACGTGCTT |
| | | GACAAGCGCCGCGGCAGAGATCCCGAGATGGGTGGAAAGCCG |
| | | CGGCGGAAGAATCCGCAGGAAGGGCTCTACAACGAGCTCCAG |
| | | AAGGACAAGATGGCCGAAGCCTACAGCGAAATCGGGATGAAG |
| | | GGCGAAAGACGCCGGGGAAAAGGACACGACGGACTGTACCAG |
| | | GGGTTGTCGACCGCGACCAAGGACACCTACGACGCCCTGCAT |
| | | ATGCAAGCCTTGCCGCCGAGATGA |
| 12 | BZ CAR | MLLLVTSLLLCELPHPAFLLIPEIVMTQSPATLSVSPGERAT |
| | nucleic | LSCRASQSVNSNLAWYQQKPGQAPRLLIYGASTRATGIPARF |
| | acid | SGSGSGTEFTLTISSLQSEDFAVYYCQQYNNWPFTFGPGTKV |
| | sequence | DIKGGGGSGGGGSGGGGSGGGGSQVQLQQSGPGLVKPSQTLS |
| | STEAP2 | LTCAISGDSVSRNSAVWNWIRQSPSRGLEWLGRTYYRSKWYN |
| | BZ CAR | DYAVSVKSRITINPDTSKNQFSLQVNSVTPEDTAVYYCARGL |
| | amino | LQNNFYYYMDVWGKGTTVTVSSGSESKYGPPCPPCPFWVLVV |
| | acid | VGGVLACYSLLVTVAFIIFWVKRGRKKLLYIFKQPFMRPVQT |
| | sequence | TQEEDGCSCRFPEEEEGGCELRVKFSRSADAPAYQQGQNQLY |
| | | NELNLGRREEYDVLDKRRGRDPEMGGKPRRKNPQEGLYNELQ |
| | | KDKMAEAYSEIGMKGERRRGKGHDGLYQGLSTATKDTYDALH |
| | | MQALPPR |
| 13 | T2A | GSGEGRGSLLTCGDVEENPGP |
| 14 | Peptide | ATGCTGCTCCTTGTCACATCACTGCTGCTCTGCGAACTGCCC |
| | T2A | CACCCTGCATTCCTCCTGATCCCCGAGATTGTGATGACCCAG |
| | STEAP2 | AGCCCTGCAACTCTGAGCGTGTCACCCGGAGAAAGGGCCACT |
| | BZ CAR- | CTGTCGTGTCGAGCATCGCAGTCCGTGAACTCCAATCTCGCC |
| | T2A- | TGGTACCAGCAGAAGCCTGGGCAGGCCCCGAGGCTGCTCATC |
| | Dominant- | TACGGTGCCTCCACGAGAGCCACGGGAATTCCAGCGCGCTTT |
| | negative | AGCGGATCCGGCTCGGGAACCGAGTTCACCCTTACCATCTCA |
| | TGF-β | TCGCTGCAATCCGAAGATTTCGCCGTGTATTACTGTCAACAG |
| | receptor | TACAACAACTGGCCGTTCACCTTTGGCCCGGGAACTAAGGTC |
| | type 2 | GACATCAAGGGCGGCGGGGGCTCTGGGGGTGGCGGAAGCGGC |
| | nucleic | GGCGGCGGATCCGGTGGCGGCGGAAGCCAAGTGCAGCTGCAG |
| | acid | CAGTCCGGACCCGGACTCGTGAAGCCGTCCCAGACTCTGTCC |
| | sequence | CTGACTTGCGCGATTTCCGGCGATTCCGTGTCCCGCAACTCC |
| | | GCTGTGTGGAACTGGATCCGGCAGTCGCCTTCGAGAGGACTG |
| | | GAGTGGCTGGGACGGACCTACTACCGCTCAAAATGGTATAAC |
| | | GACTATGCTGTGTCCGTCAAGAGCCGCATCACCATTAACCCC |
| | | GATACCTCCAAGAACCAGTTCAGTCTGCAAGTCAACAGCGTG |
| | | ACTCCTGAGGACACCGCCGTGTACTACTGCGCCCGGGGTCTG |
| | | CTGCAAAACAACTTCTACTACTACATGGACGTCTGGGGAAAG |
| | | GGAACTACTGTGACCGTGTCCTCCGGCTCCGAATCAAAATAC |
| | | GGTCCGCCATGCCCACCGTGCCCCTTCTGGGTGCTCGTGGTC |
| | | GTCGGAGGGGTTCTGGCCTGCTACTCCCTGCTGGTCACCGTG |
| | | GCGTTTATCATCTTCTGGGTGAAGCGGGGAAGGAAGAAGCTA |
| | | CTGTACATTTTCAAGCAGCCTTTCATGCGGCCTGTGCAGACC |
| | | ACCCAGGAAGAGGACGGCTGTTCCTGCCGGTTCCCCGAGGAA |
| | | GAGGAAGGGGGTTGCGAGCTGCGCGTGAAGTTCAGCAGGAGC |
| | | GCTGATGCCCCAGCGTACCAACAGGGGCAAAACCAGTTGTAC |
| | | AACGAACTGAACCTTGGTCGGCGCGAAGAGTACGACGTGCTT |
| | | GACAAGCGCCGCGGCAGAGATCCCGAGATGGGTGGAAAGCCG |
| | | CGGCGGAAGAATCCGCAGGAAGGGCTCTACAACGAGCTCCAG |
| | | AAGGACAAGATGGCCGAAGCCTACAGCGAAATCGGGATGAAG |
| | | GGCGAAAGACGCCGGGGAAAAGGACACGACGGACTGTACCAG |
| | | GGGTTGTCGACCGCGACCAAGGACACCTACGACGCCCTGCAT |
| | | ATGCAAGCCTTGCCGCCGAGAGGATCCGGAGAGGGGAGGGGA |
| | | AGCCTCCTCACTTGCGGCGATGTGGAGGAAAACCCGGGTCCT |
| | | ATGGGACGCGGGCTGCTTCGAGGACTCTGGCCACTTCATATC |
| | | GTGTTGTGGACTCGCATCGCTTCAACCATTCCGCCGCACGTG |
| | | CAGAAGTCCGTGAACAATGACATGATCGTGACCGACAACAAC |
| | | GGTGCAGTGAAGTTCCCACAGCTGTGCAAGTTCTGCGATGTC |
| | | AGATTCAGCACTTGCGACAACCAGAAGTCCTGCATGTCAAAC |
| | | TGCTCCATCACCTCCATCTGCGAGAAGCCTCAAGAGGTCTGC |
| | | GTGGCCGTGTGGCGGAAGAACGACGAGAACATCACCCTGGAA |
| | | ACCGTGTGCCACGATCCGAAGCTGCCTTATCACGACTTCATT |
| | | CTGGAAGATGCCGCCTCGCCCAAGTGTATCATGAAAGAAAAG |
| | | AAAAAGCCCGGAGAAACGTTCTTCATGTGCTCGTGTAGCTCC |
| | | GACGAGTGCAACGACAACATTATCTTTAGCGAAGAGTACAAC |
| | | ACTTCCAACCCTGACCTCCTGCTCGTGATTTTTCAAGTCACC |
| | | GGCATTTCCCTGCTGCCCCCGCTGGGCGTGGCGATCTCGGTG |
| | | ATCATTATCTTCTACTGTTACCGGGTCAATAGGCAG |
| 15 | STEAP2 | MLLLVTSLLLCELPHPAFLLIPEIVMTQSPATLSVSPGERAT |
| | BZ CAR- | LSCRASQSVNSNLAWYQQKPGQAPRLLIYGASTRATGIPARF |
| | T2A- | SGSGSGTEFTLTISSLQSEDFAVYYCQQYNNWPFTFGPGTKV |
| | Dominant | DIKGGGGSGGGGSGGGGSGGGGSQVQLQQSGPGLVKPSQTLS |
| | negative | LTCAISGDSVSRNSAVWNWIRQSPSRGLEWLGRTYYRSKWYN |
| | TGF-β | DYAVSVKSRITINPDTSKNQFSLQVNSVTPEDTAVYYCARGL |
| | receptor | LQNNFYYYMDVWGKGTTVTVSSGSESKYGPPCPPCPFWVLVV |
| | type 2 | VGGVLACYSLLVTVAFIIFWVKRGRKKLLYIFKQPFMRPVQT |
| | amino | TQEEDGCSCRFPEEEEGGCELRVKFSRSADAPAYQQGQNQLY |
| | acid | NELNLGRREEYDVLDKRRGRDPEMGGKPRRKNPQEGLYNELQ |
| | sequence | KDKMAEAYSEIGMKGERRRGKGHDGLYQGLSTATKDTYDALH |
| | | MQALPPRGSGEGRGSLLTCGDVEENPGPMGRGLLRGLWPLHI |
| | | VLWTRIASTIPPHVQKSVNNDMIVTDNNGAVKFPQLCKFCDV |
| | | RFSTCDNQKSCMSNCSITSICEKPQEVCVAVWRKNDENITLE |
| | | TVCHDPKLPYHDFILEDAASPKCIMKEKKKPGETFFMCSCSS |
| | | DECNDNIIFSEEYNTSNPDLLLVIFQVTGISLIPPLGVAISV |
| | | IIIFYCYRVNRQ |
| 16 | STEAP2 | EIVMTQSPATLSVSPGERATLSCRASQSVNSNLAWYQQKPGQ |
| | SCFV | APRLLIYGASTRATGIPARFSGSGSGTEFTLTISSLQSEDFA |
| | amino | VYYCQQYNNWPFTFGPGTKVDIKGGGGSGGGGSGGGGSGGGG |
| | acid | SQVQLQQSGPGLVKPSQTLSLTCAISGDSVSRNSAVWNWIRQ |
| | sequence | SPSRGLEWLGRTYYRSKWYNDYAVSVKSRITINPDTSKNQFS |
| | | LQVNSVTPEDTAVYYCARGLLQNNFYYYMDVWGKGTTVTVSS |
| 17 | 4-1BB | KRGRKKLLYIFKQPFMRPVQTTQEEDGCSCRFPEEEEGGCEL |
| | Activation | |
| | Domain | |
| | (B | |
| | domain) | |
| 18 | CD3zeta | RVKFSRSADAPAYQQGQNQLYNELNLGRREEYDVLDKRRGRD |
| | Domain | PEMGGKPRRKNPQEGLYNELQKDKMAEAYSEIGMKGERRRGK |
| | (Z | GHDGLYQGLSTATKDTYDALHMQALPPR |
| | domain) | |
| 19 | CD28 Co- | RSKRSRLLHSDYMNMTPRRPGPTRKHYQPYAPPRDFAAYRS |
| | stimulatory | |
| | Domain | |
| 20 | CD28 | FWVLVVVGGVLACYSLLVTVAFIIFWV |
| | Transmembrane | |
| | Domain | |
| 21 | IgG4P | ESKYGPPCPPCP |
| | Hinge | |
| | (S228P) | |
| 22 | STEAP2 | GAGATTGTGATGACCCAGAGCCCTGCAACTCTGAGCGTGTCA |
| | SCFV | CCCGGAGAAAGGGCCACTCTGTCGTGTCGAGCATCGCAGTCC |
| | nucleic | GTGAACTCCAATCTCGCCTGGTACCAGCAGAAGCCTGGGCAG |
| | acid | GCCCCGAGGCTGCTCATCTACGGTGCCTCCACGAGAGCCACG |
| | sequence | GGAATTCCAGCGCGCTTTAGCGGATCCGGCTCGGGAACCGAG |
| | | TTCACCCTTACCATCTCATCGCTGCAATCCGAAGATTTCGCC |
| | | GTGTATTACTGTCAACAGTACAACAACTGGCCGTTCACCTTT |
| | | GGCCCGGGAACTAAGGTCGACATCAAGGGCGGCGGGGGCTCT |
| | | GGGGGTGGCGGAAGCGGCGGCGGCGGATCCGGTGGCGGCGGA |
| | | AGCCAAGTGCAGCTGCAGCAGTCCGGACCCGGACTCGTGAAG |
| | | CCGTCCCAGACTCTGTCCCTGACTTGCGCGATTTCCGGCGAT |
| | | TCCGTGTCCCGCAACTCCGCTGTGTGGAACTGGATCCGGCAG |
| | | TCGCCTTCGAGAGGACTGGAGTGGCTGGGACGGACCTACTAC |
| | | CGCTCAAAATGGTATAACGACTATGCTGTGTCCGTCAAGAGC |
| | | CGCATCACCATTAACCCCGATACCTCCAAGAACCAGTTCAGT |
| | | CTGCAAGTCAACAGCGTGACTCCTGAGGACACCGCCGTGTAC |
| | | TACTGCGCCCGGGGTCTGCTGCAAAACAACTTCTACTACTAC |
| | | ATGGACGTCTGGGGAAAGGGAACTACTGTGACCGTGTCCTCC |

The practice of the present disclosure will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); Crooks, Antisense drug Technology: Principles, strategies and applications, 2^{nd} Ed. CRC Press (2007) and in Ausubel et al. (1989) Current Protocols in Molecular Biology (John Wiley and Sons, Baltimore, Md.).

The following examples are offered by way of illustration and not by way of limitation.

### EXAMPLES

### Example 1: Effect of Androgen Receptor Antagonist on STEAP2-Positive Prostate Cancer

Six-transmembrane epithelial antigen of the prostate-2 (STEAP2) expression is increased in prostate cancer when compared to normal prostate, suggesting STEAP2 may drive prostate cancer progression. STEAP2 drives aggressive prostate cancer traits by promoting proliferation, migration and invasion, with downstream targets, including the metalloproteases MMP3, MMP10, MMP13, and MMP7, which are known as being associated with driving invasion and metastatic potential (Burnell S et al. Sci. Rep. 2018; 8:6252). STEAP2 overexpression increases the migration and invasion abilities of prostate cancer cells, and is associated with advanced cancer stage and histologic grading (Whiteland H et al. Clin. Exp. Metastasis. 2014; 31(8):909-20).

Androgen receptor antagonists (antiandrogens) are drugs used to treat hormonal-based syndromes and prostate cancer. Current drugs for prostate cancer include flutamide, bicalutamide, nilutamide, enzalutamide and ARN-509. Each of these inhibitors binds to the ligand-binding domain (LBD) of the androgen receptor. This is the same site that the natural physiological steroids testosterone (TES) and dihydrotestosterone (DHT) bind. The drugs work by competing with the natural hormones for binding to the LBD and, as a result, lessening activation of the receptor.

In order to determine the effects of androgen receptor antagonists on prostate cancer cell lines in the context of STEAP2, LuCaP prostate cancer patient-derived xenografts (PDX) were utilized (see, Nguyen H M et al. Prostate. 2017; 77(6): 654-671, which is incorporated herein by reference). Specifically, LuCaP73, LuCaP70, and LuCaP86.2 PDX models were evaluated for their response to enzalutamide treatment. As described in Nguyen, LuCaP73 PDX model represents more castration sensitive model (+++) then LuCaP70 PDX model (++), whereas LuCaP86.2 PDX model shows the least responsiveness to castration therapy.

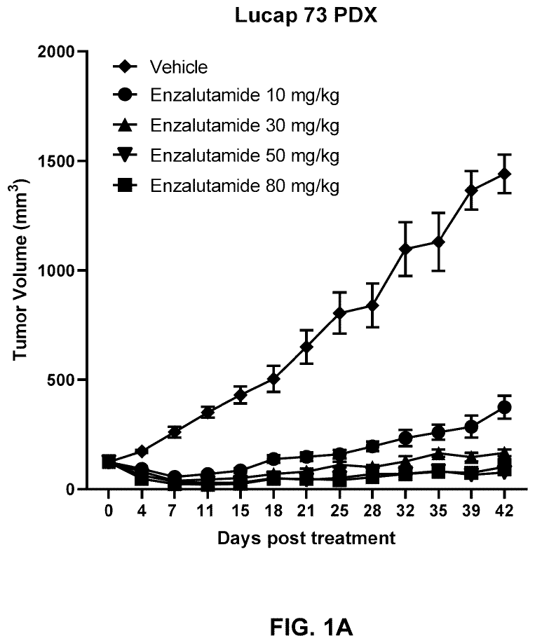

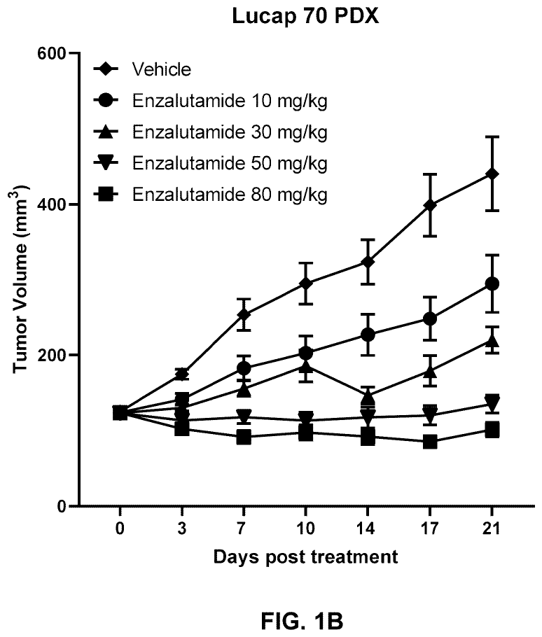

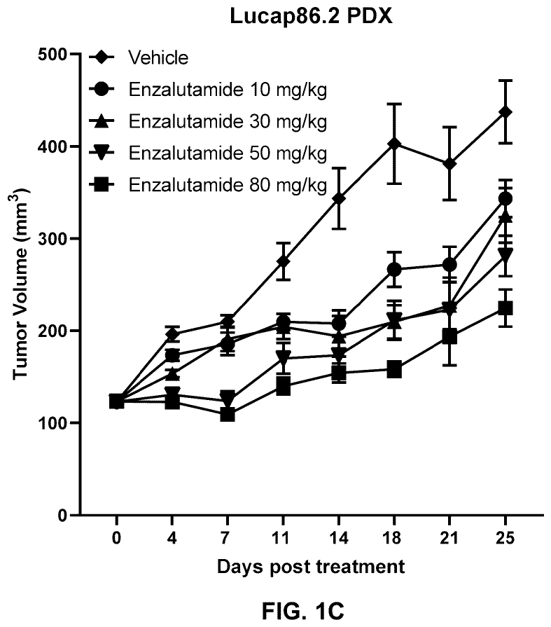

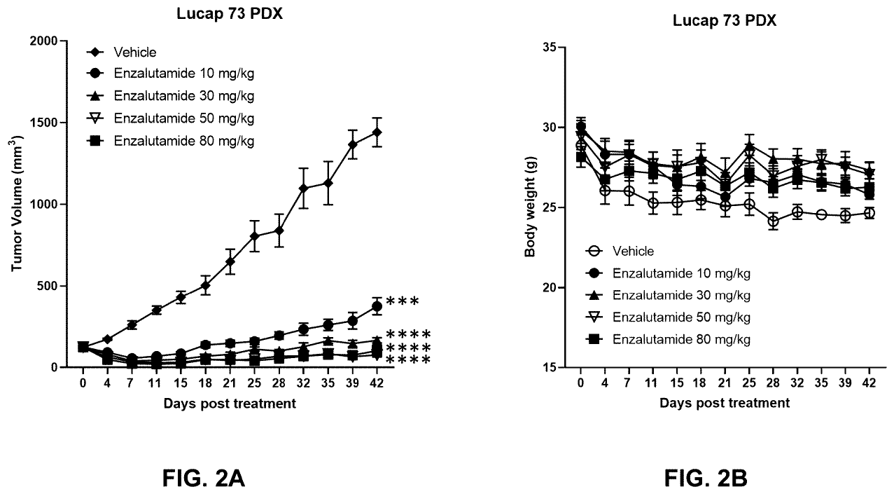

As shown in FIGS. 1A-1C, three LuCaP prostate cancer patient-derived xenograft (PDX) models were evaluated following enzalutamide treatment across 42 days. Enzalutamide was administered orally, once a day. The drug vehicle consisted of 5% DMSO, 1% carboxymethyl cellulose, and 0.1% Tween 80. Tumor volume was measured for up to 42 days following treatment with either a vehicle control or enzalutamide (10, 30, 50, or 80 mg/kg doses) in LuCaP73 (FIG. 1A), LuCaP70 (FIG. 1B), and LuCaP86.2 (FIG. 1C) PDX models (8 mice per group). Enzalutamide treatment resulted in a dose-dependent decrease in tumor volume, with the most significant decrease seen in the most castration sensitive LuCaP73 PDX model (FIG. 2A) (*** indicates p<0.001 and **** indicates p<0.0001). Body weight of mice implanted with LuCaP73 cells was also slightly improved following enzalutamide treatment (FIG. 2B).

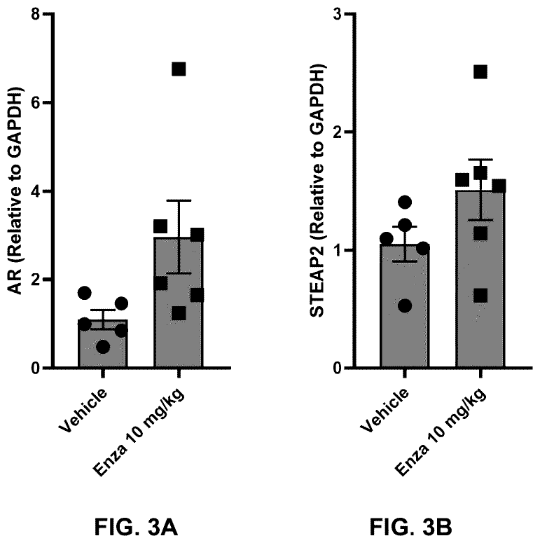

FIGS. 3A-3B show LuCaP73 tumor expression of androgen receptor (AR; FIG. 3A) and STEAP2 (FIG. 3B) at day 42 following treatment with vehicle or 10 mg/kg enzalutamide (Enza). AR and STEAP2 expression levels were normalized to GAPDH levels. Following treatment with enzalutamide, both AR and STEAP2 expression increased in LuCaP73 tumors.

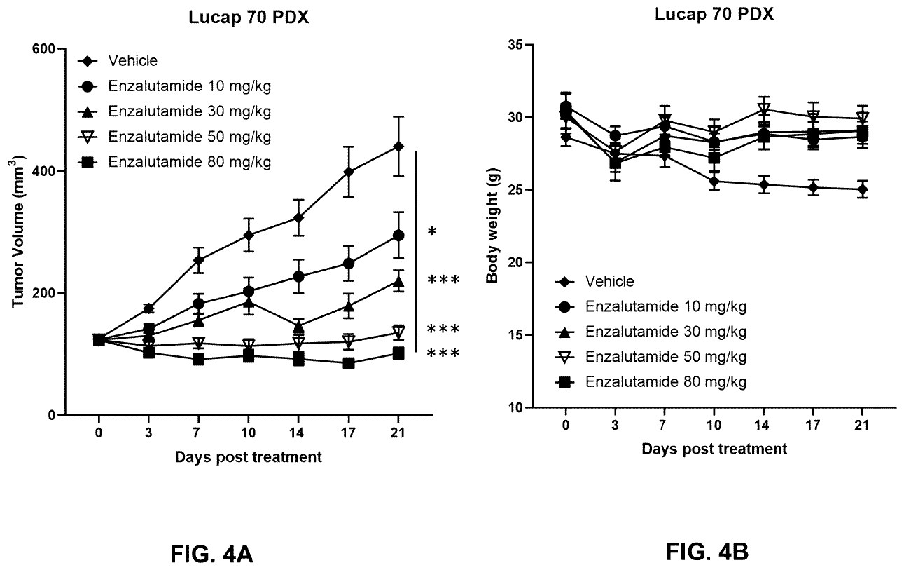

As shown in FIG. 4A, the castration-sensitive LuCaP70 PDX model also had significant decreases in tumor volume to varying doses of enzalutamide treatment over time. Tumor volume was measured for 21 days following treatment with either a vehicle control or enzalutamide (10, 30, 50, or 80 mg/kg doses). *** indicates p<0.001 and * indicates p<0.05. FIG. 4B shows the body weight of a LuCaP-70 PDX model following treatment with either a vehicle control or enzalutamide (10, 30, 50, or 80 mg/kg doses), indicating an improvement in body weight with enzalutamide treatment.

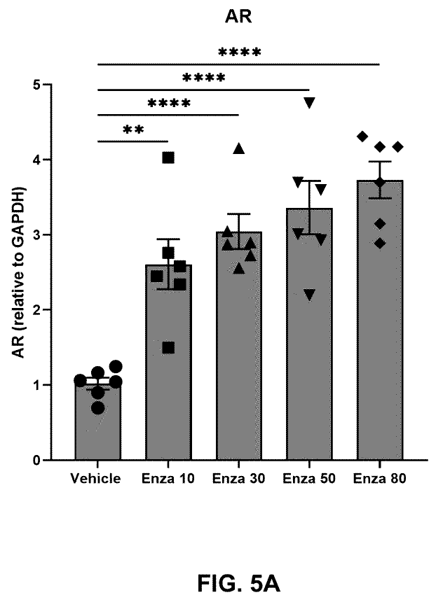

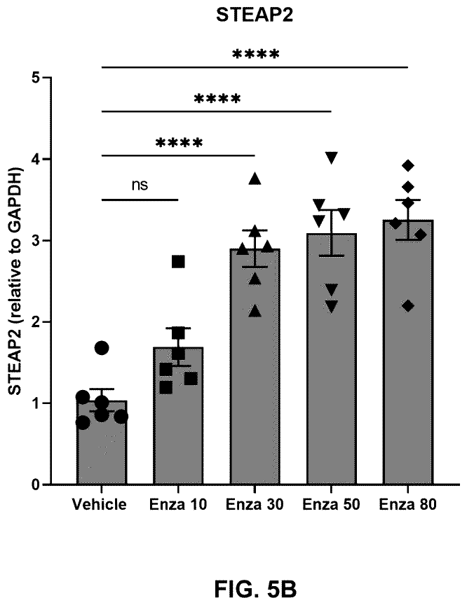

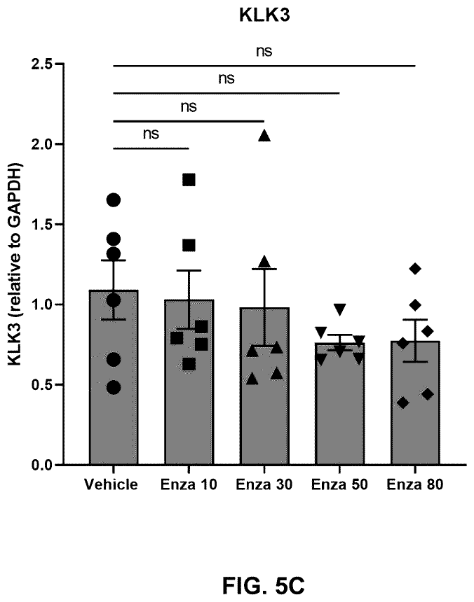

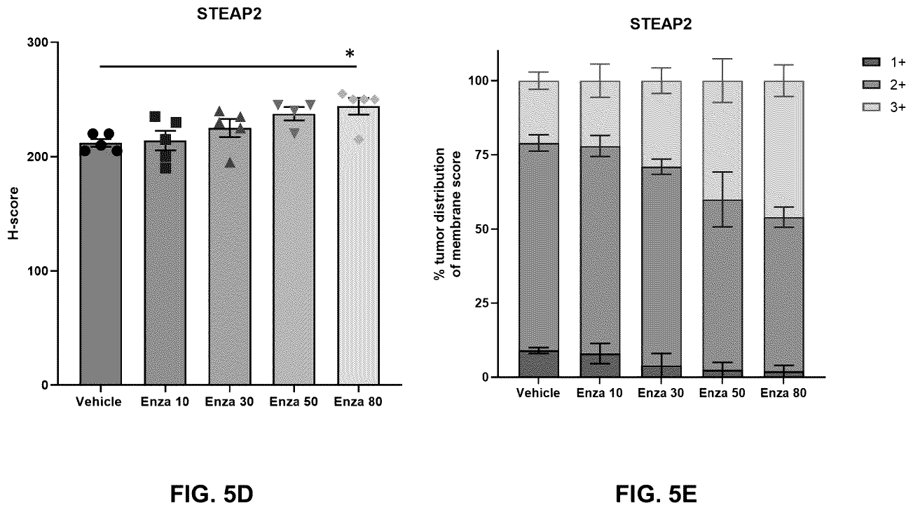

FIGS. 5A-5C show LuCaP70 tumor expression of androgen receptor (AR; FIG. 5A), STEAP2 (FIG. 5B), and KLK3 (FIG. 5C) at day 21 following treatment with vehicle or enzalutamide (10, 30, 50, or 80 mg/kg doses). **** indicates p<0.0001; ** indicates p<0.01; and ns indicates not significant. A significant dose-dependent increase in AR and STEAP2 levels is seen, while KLK3 did not have significant changes in expression levels.

FIGS. 5D-5E show STEAP2 expression levels in LuCaP70 tumors based on immunohistochemistry (IHC) using an anti-STEAP2 antibody and calculated intensity of tumor membrane staining and percentage of cells stained (H-score; FIG. 5D) and by percent membrane staining in tumors (FIG. 5E) (* indicates p<0.05). The highest dose of enzalutamide (80 mg/kg) had a significant effect (p<0.05) on the STEAP2 H-score, indicating an increase in STEAP2 membrane staining. Enzalutamide treatment had a dose dependent effect on STEAP2% tumor distribution of membrane score (e.g., vehicle treated tumors resulted in approximately 8.75% STEAP2 1+, approximately 72.5% STEAP2 2+ and approximately 18.75% STEAP2 3+, whereas enzalutamide 80 mg/kg treatment resulted in approximately 2% STEAP2 1+, approximately 52% STEAP2 2+ and approximately 46% STEAP2 3+ distribution. These results indicate an effect of enzalutamide treatment on STEAP2 membrane expression.

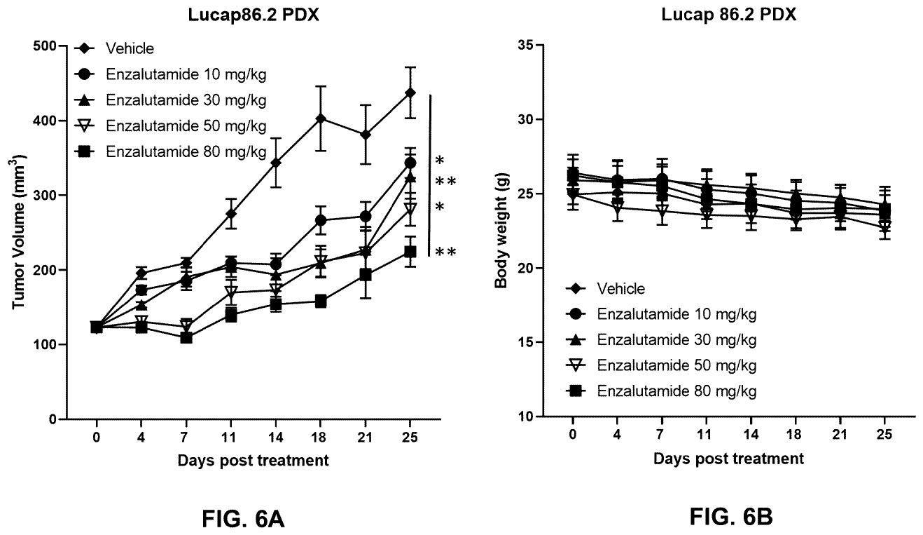

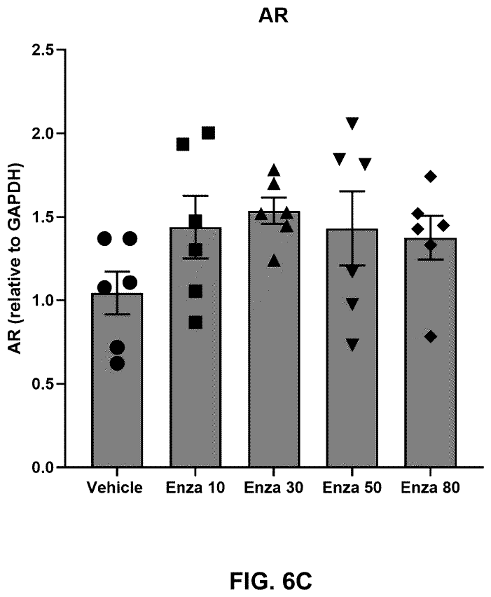

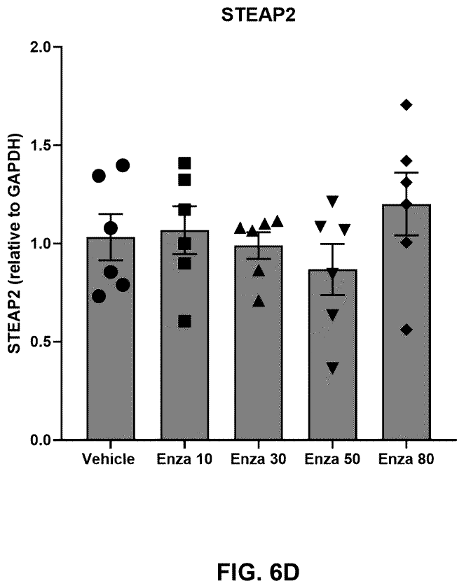

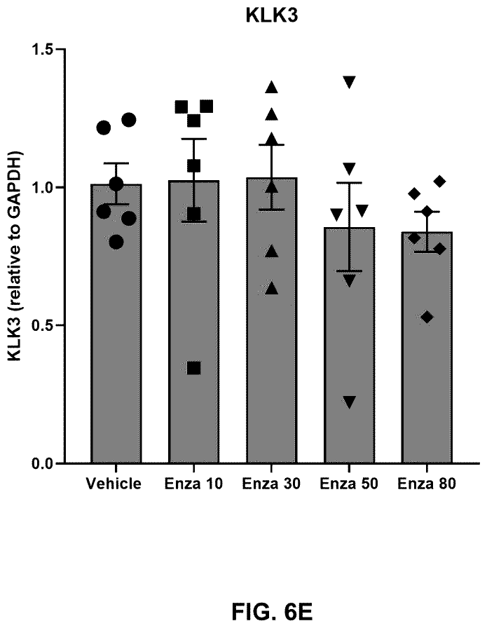

As shown in FIG. 6A, the response of the castration-resistant LuCaP86.2 PDX model to varying doses of enzalutamide treatment over time was slightly less significant than the responses observed in LuCaP73 and LuCaP70 PDX models. Tumor volume was measured for 25 days following treatment with either a vehicle control or enzalutamide (10, 30, 50, or 80 mg/kg doses). ** indicates P<0.01, and * indicates p<0.05. FIG. 6B shows the body weight of a LuCaP 86.2 PDX model following treatment with either a vehicle control or enzalutamide (10, 30, 50, or 80 mg/kg doses), where there were no significant variations in body weight with enzalutamide treatment compared to treatment with vehicle.

FIGS. 6C-6E show LuCaP86.2 tumor expression of androgen receptor (AR; FIG. 6C), STEAP2 (FIG. 6D), and KLK3 (FIG. 6E) at day 25 following treatment with vehicle or enzalutamide (10, 30, 50, or 80 mg/kg doses). No significant differences were seen in AR, STEAP2, or KLK3 expression levels at most dosages of enzalutamide, with only a minor increase in STEAP2 levels at the 80 mg/kg dose of enzalutamide. The impaired response to enzalutamide may be related to the AR^{V567} variant expressed in LuCaP 86.2 tumors, which is a constitutively active splice variant of AR.

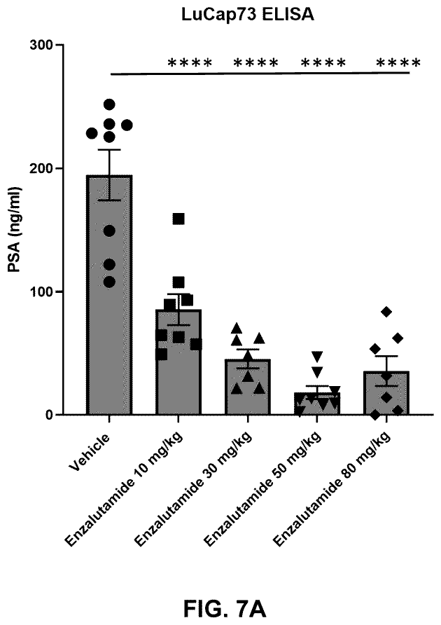

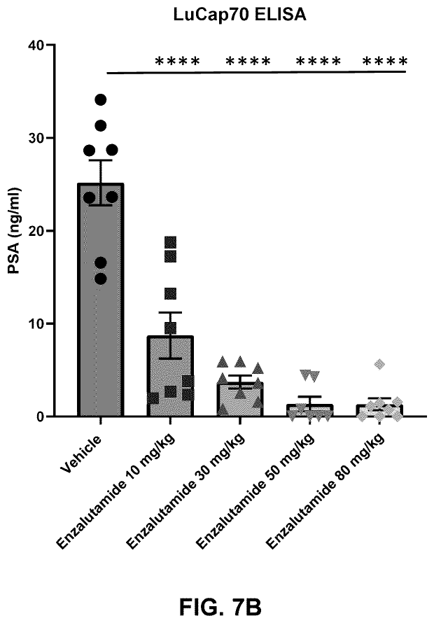

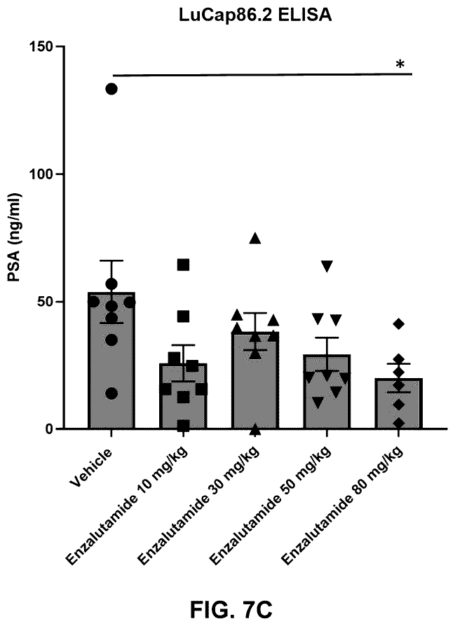

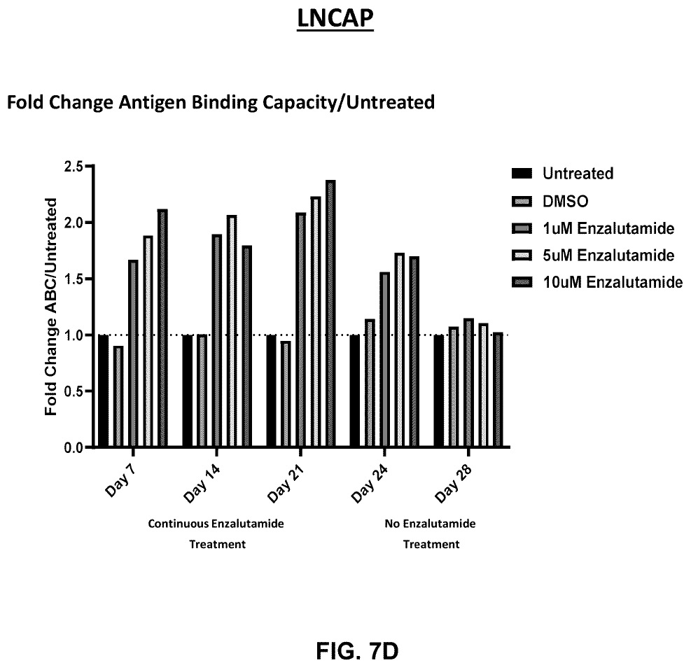

Serum levels of prostate-specific antigen (PSA) in castration-sensitive LuCaP PDX models was measured following enzalutamide treatment. LuCaP73 (FIG. 7A), LuCaP70 (FIG. 7B), and LuCaP86.2 (FIG. 7C) PDX models were treated with either vehicle or enzalutamide (10, 30, 50, or 80 mg/kg doses) and serum PSA levels were determined by ELISA at the end of the study (LuCaP73=Day 42, LuCaP70=Day 21, and LuCaP86.2=Day 25). **** indicates p<0.0001 and * indicates p<0.05. A significant decreased in serum PSA was found across all dosages of enzalutamide in the mice implanted with castration-sensitive LuCaP73 and LuCaP70 tumors, while only the 80 mg/kg dose of enzalutamide led to a significant decrease in serum PSA in the mice implanted with castration-resistant LuCaP86.2 tumors.

STEAP2 receptor density and antigen binding capacity (ABC) after continuous enzalutamide exposure was also measured in LNCAP cells over a 28 day period. The effect on STEAP2 mean fluorescence intensity (MFI) after continuous enzalutamide treatment increases STEAP2 receptor density levels up to 21 days in a full length AR castration sensitive LNCAP cell line. After day 21, upon enzalutamide removal, STEAP2 levels return to baseline level.

These results indicate that castration-sensitive prostate cancer PDX models are more readily targeted by androgen receptor antagonists such as enzalutamide. Additionally, enzalutamide treatment was found to significantly increase STEAP2 expression in castration sensitive prostate cancer PDX models.

### Example 2: In Vivo Efficacy of Anti-STEAP2 CAR T and Androgen Receptor Antagonist Combination Therapy in a Mouse Model

As described in Example 1, androgen receptor antagonist treatment (e.g., enzalutamide) of castration-sensitive prostate cancer cells led to a significant increase in STEAP2 expression. AZD0754 is an anti-STEAP2 chimeric antigen receptor (CAR) T cell (clone 40A3) expressing a dominant-negative form of TFGβRII (dnTFGβRII) for improved tumor microenvironment activity. Using a LuCaP73 PDX model in NSG-MHC I/II Double Knock Out mice (also known as NSG-(K^{b}D^{b})^{null} (IA)^{null}, NSG-(K^{b} D^{b})^{null} (IA^{null}); The Jackson Laboratory; Strain #025216), AZD0754 treatment was evaluated for therapeutic efficacy.

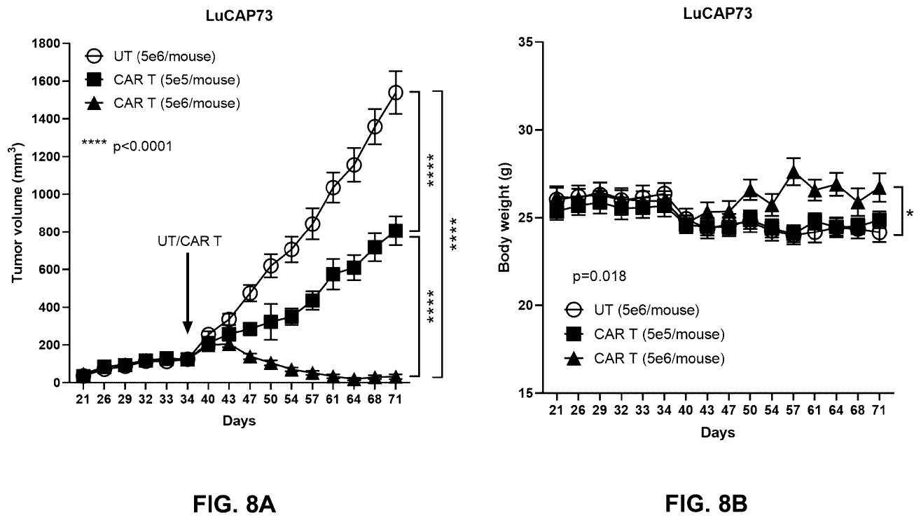

Tumor volume (FIG. 8A) and body weight (FIG. 8B) was measured over time for mice treated with untransduced T cells (UT; 5×10⁶ cells/mouse), 40A3 dnTFGβRII (i.e., AZD0754) CAR T cells (CAR T; 5×10⁵ cells/mouse), or 40A3 dnTFGβRII CAR T cells (CAR T; 5×10⁶ cells/mouse). UT/CAR T treatment was administered at the indicated point in time (day 34). N=12 for each treatment group. **** indicates p<0.0001 and * indicates p<0.05. A significant decrease in tumor volume was found following AZD0754 treatment at either 5×10⁵ or 5×10⁶ cells/mouse, while a significant increase in body weight was observed only with the 5×10⁶ cells/mouse dose of AZD0754.

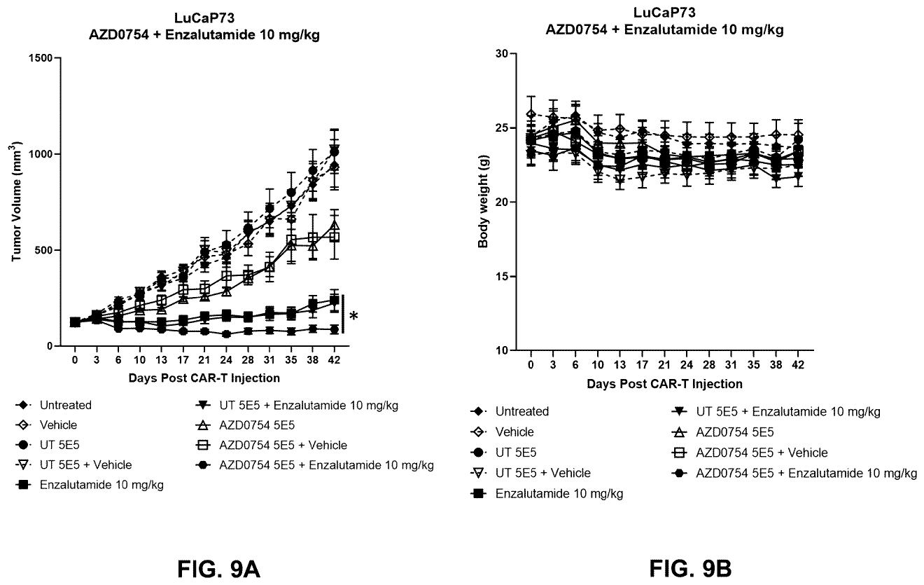

FIGS. 9A-9B show the response of AZD0754 CAR T cell and enzalutamide combination therapy in a LuCaP73 PDX model. Tumor volume (FIG. 9A) and body weight (FIG. 9B) was measured over time for untreated mice, mice with vehicle control, mice treated with untransduced T cell (UT; 5×10⁵ cells/mouse), mice treated with untransduced T cell (UT; 5×10⁵ cells/mouse) and vehicle control, mice treated with enzalutamide (10 mg/kg), mice treated with untransduced T cells (UT; 5×10⁵ cells/mouse) and enzalutamide (10 mg/kg), mice treated with AZD0754 (5×10⁵ cells/mouse), mice treated with AZD0754 (5×10⁵ cells/mouse) and vehicle control, and mice treated with AZD0754 (5×10⁵ cells/mouse) and enzalutamide (10 mg/kg). N=9 for each treatment group. * indicates p<0.05. A significant decrease in tumor volume was observed in the AZD0754+enzalutamide combination therapy group in comparison to enzalutamide only treated group.

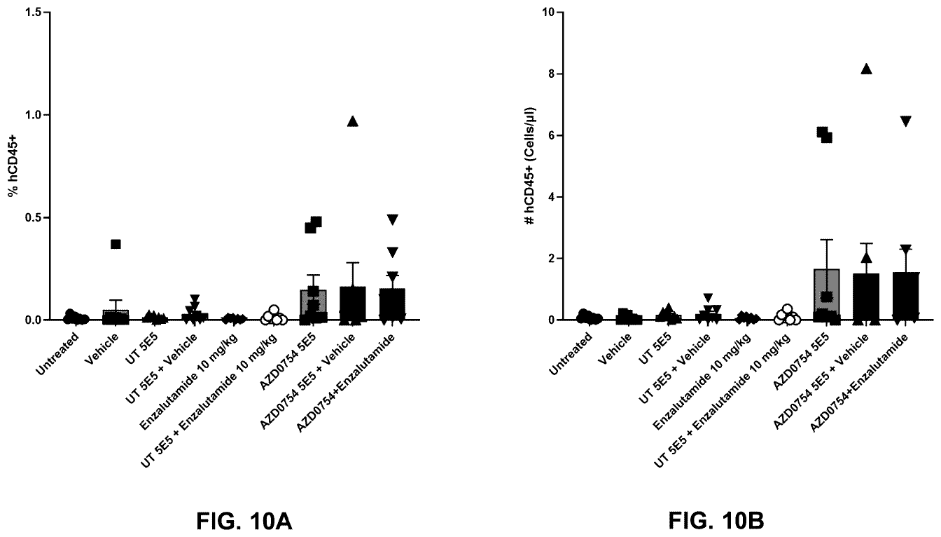

FIGS. 10A-10B show the percentage (FIG. 10A) and cell number (FIG. 10B) of AZD0754 CAR-T cells at day 42 in the blood of untreated mice, mice with vehicle control, mice treated with untransduced T cell (UT; 5×10⁵ cells/mouse), mice treated with untransduced T cell (UT; 5×10⁵ cells/mouse) and vehicle control, mice treated with enzalutamide (10 mg/kg), mice treated with untransduced T cells (UT; 5×10⁵ cells/mouse) and enzalutamide (10 mg/kg), mice treated with AZD0754 (5×10⁵ cells/mouse), mice treated with AZD0754 (5×10⁵ cells/mouse) and vehicle control, and mice treated with AZD0754 (5×10⁵ cells/mouse) and enzalutamide (10 mg/kg). These results indicate comparable percentage and cell number of AZD0754 CAR T cells at the end of the study, indicating that the enzalutamide was not having a negative effect on CAR T cell viability.

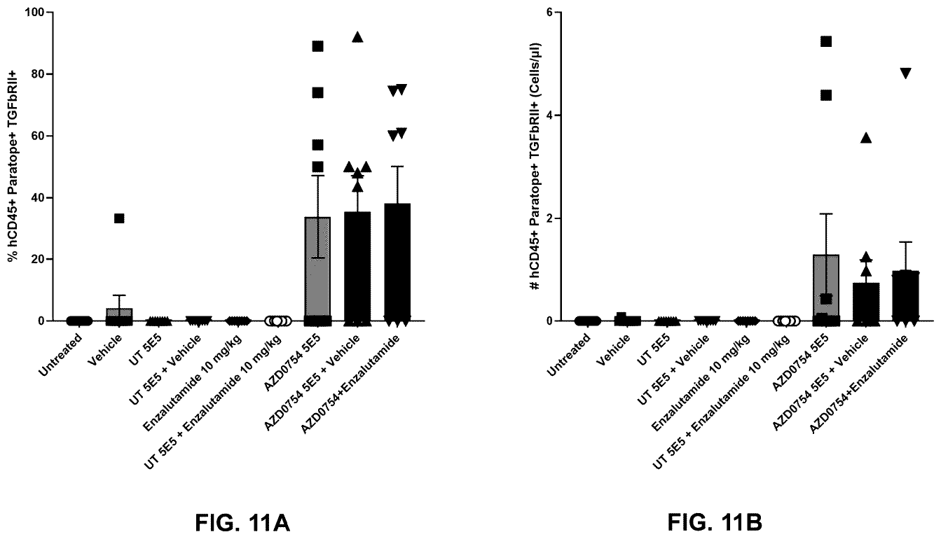

FIGS. 11A-11B show the percentage (FIG. 11A) and cell number (FIG. 11B) of hCD45+ Paratope+ TGFβRII+ CAR-T cells at day 42 in the blood of untreated mice, mice with vehicle control, mice treated with untransduced T cell (UT; 5×10⁵ cells/mouse), mice treated with untransduced T cell (UT; 5×10⁵ cells/mouse) and vehicle control, mice treated with enzalutamide (10 mg/kg), mice treated with untransduced T cells (UT; 5×10⁵ cells/mouse) and enzalutamide (10 mg/kg), mice treated with AZD0754 (5×10⁵ cells/mouse), mice treated with AZD0754 (5×10⁵ cells/mouse) and vehicle control, and mice treated with AZD0754 (5×10⁵ cells/mouse) and enzalutamide (10 mg/kg). These results indicate comparable percentage and cell number of hCD45⁺ Paratope⁺ TGFβRII⁺ CAR T cells at the end of the study, indicating that the enzalutamide was not having a negative effect on CAR T cell viability and phenotype.

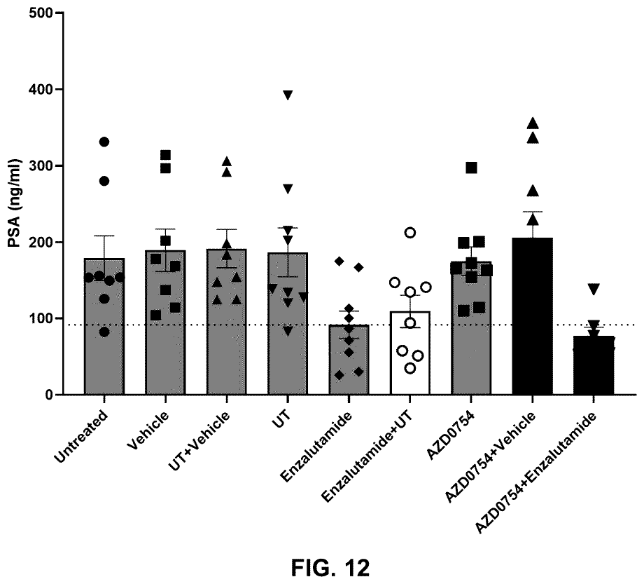

FIG. 12 shows the levels of serum PSA at day 42 in untreated mice, mice with vehicle control, mice treated with untransduced T cell (UT; 5×10⁵ cells/mouse), mice treated with untransduced T cell (UT; 5×10⁵ cells/mouse) and vehicle control, mice treated with enzalutamide (10 mg/kg), mice treated with untransduced T cells (UT; 5×10⁵ cells/mouse) and enzalutamide (10 mg/kg), mice treated with AZD0754 (5×10⁵ cells/mouse), mice treated with AZD0754 (5×10⁵ cells/mouse) and vehicle control, and mice treated with AZD0754 (5×10⁵ cells/mouse) and enzalutamide (10 mg/kg). The results indicate decreased levels of serum PSA following the AZD0754 and enzalutamide combination treatment, similar to or lower than enzalutamide treatment alone.

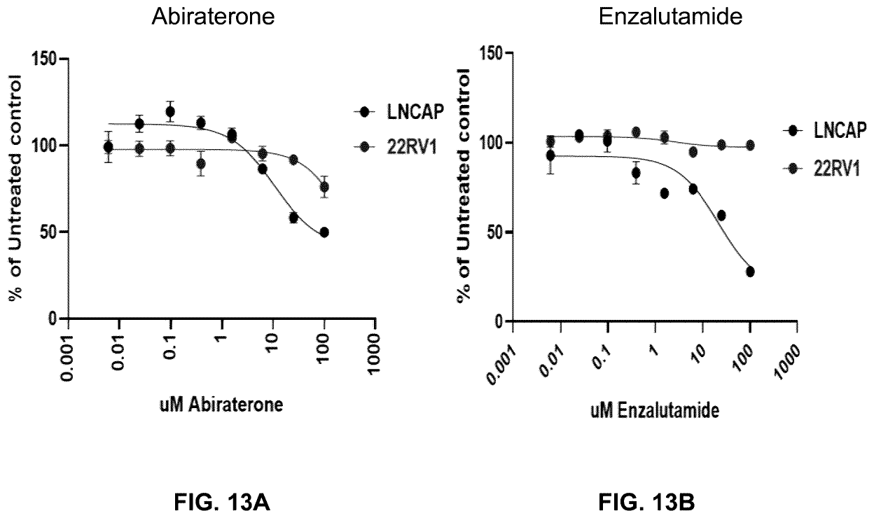

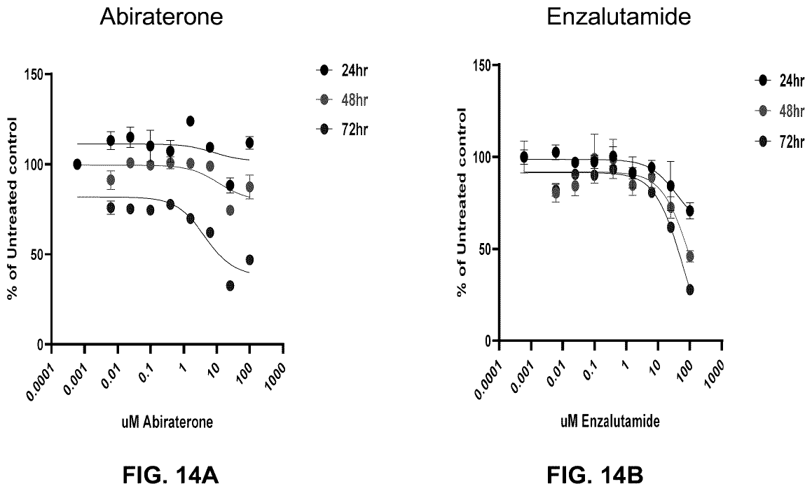

The effect of the androgen receptor antagonists abiraterone and enzalutamide were assessed on both prostate cell line and AZD0754 CAR T cell proliferation. FIGS. 13A-13B show the effect of abiraterone (FIG. 13A) and enzalutamide (FIG. 13B) treatment on LNCAP or 22RV1 prostate cell line proliferation in a dose dependent manner. FIGS. 14A-14B show the effect of abiraterone (FIG. 14A) and enzalutamide (FIG. 14B) treatment on 40A3 dnTFGβRII CAR T cell proliferation following 24, 48, or 72 hours post-treatment. LNCAP cells were found to be more sensitive to androgen receptor antagonist treatment compared to 22RV1 cells. Doses of approximately 5 uM and above of abiraterone and enzalutamide were found to suppress AZD0754 proliferation.

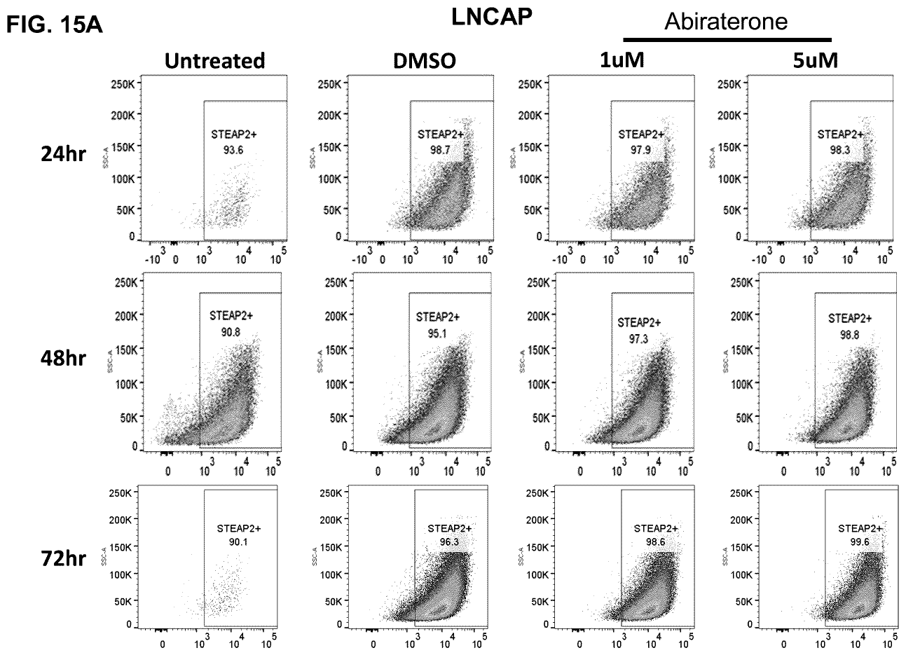

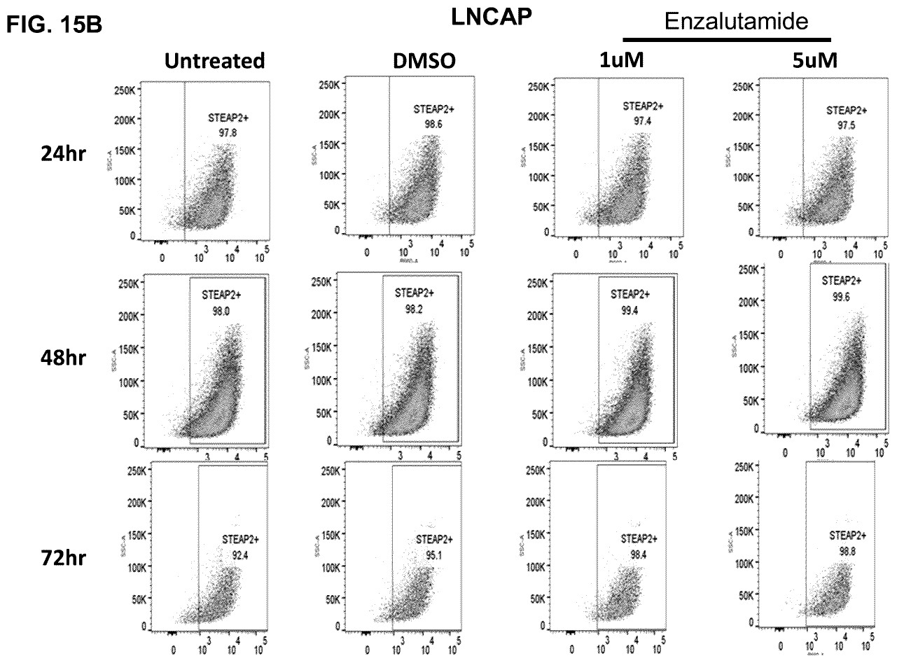

FIGS. 15A-15B show the effect of abiraterone (FIG. 15A) and enzalutamide (FIG. 15B) treatment on STEAP2 expression measured by flow cytometry in LNCAP cells following 24, 48, and 72 hours post-treatment. No effect on STEAP2 expression was seen following either abiraterone or enzalutamide treatment.

The effects of abiraterone and enzalutamide treatment on AZD0754 activity while co-cultured with LNCAP cells was assessed. LNCAP cells were pre-treated with either 0 or 5 uM of abiraterone or enzalutamide, or DMSO, for 24 hours in complete media. Then, AZD0754 or untransduced T cells were added with or without abiraterone or enzalutamide at a concentration of 5 uM. IFNγ release was also measured by harvesting the cell culture supernatant following 24 hours after CAR T cell treatment. Furthermore, gene expression analysis of downstream androgen receptor targets was performed by qPCR using LNCAP cells treated with 0 or 5 uM abiraterone or enzalutamide, or DMSO, for 72 hours in complete media. Cells were subsequently harvested and RNA extracted for qPCR analysis.

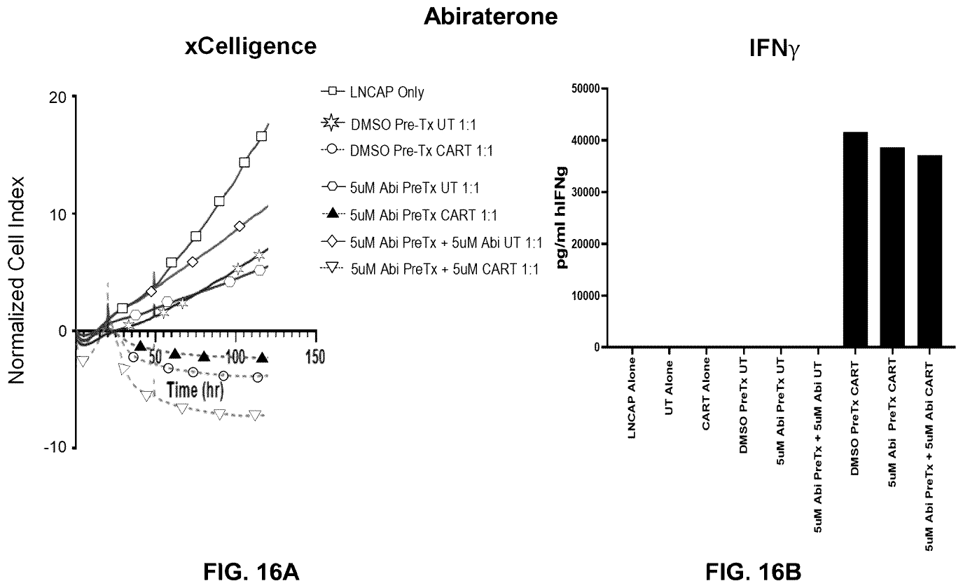

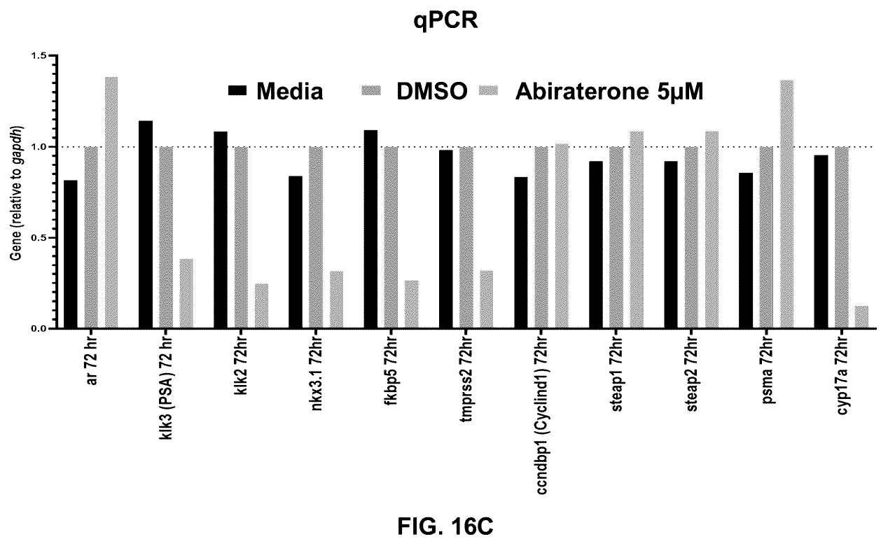

FIGS. 16A-16C show the effect of abiraterone treatment on 40A3 dnTFGβRII CAR T cell killing capacity (FIG. 16A) or IFNγ release by the CAR T cells (FIG. 16B) in co-culture with LNCAP cells. 5 uM abiraterone treatment was compared to DMSO, and CAR T cells were compared to untransduced T cells (UT), wherein the LNCAP and/or CAR T or UT cells were pre-treated (PreTx) with abiraterone. Androgen receptor inhibition in LNCAP cells treated with either media, DMSO, or 5 uM abiraterone was validated by qPCR of downstream target expression (FIG. 16C). Abiraterone did not appear to have an effect on CAR T killing capacity or IFNγ release when co-cultured with LNCAP cells.

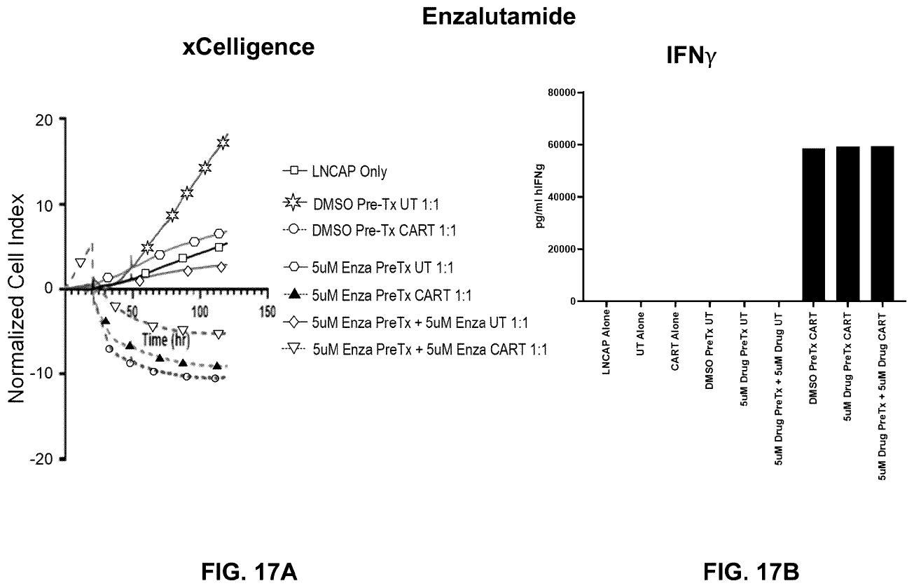

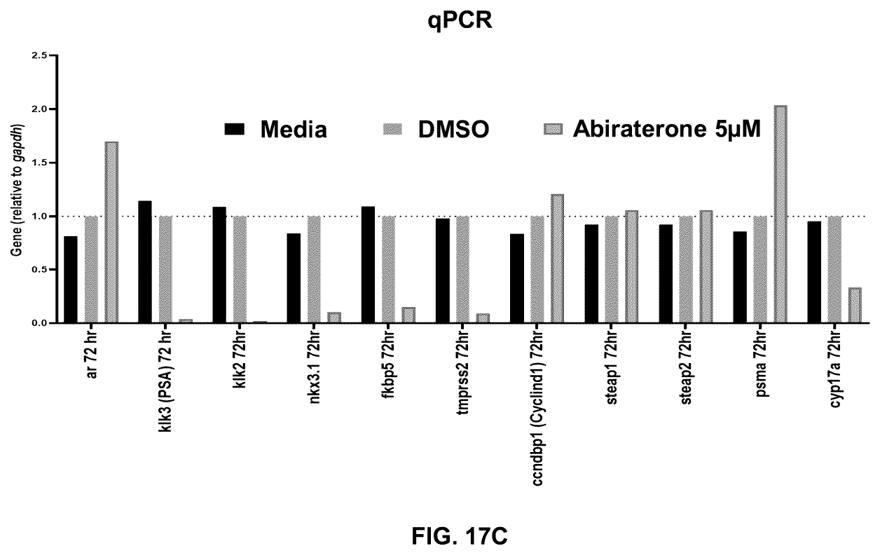

FIGS. 17A-17C show the effect of enzalutamide treatment on 40A3 dnTFGβRII CAR T cell killing capacity (FIG. 17A) or IFNγ release by the CAR T cells (FIG. 17B) in co-culture with LNCAP cells. 5 uM enzalutamide treatment was compared to DMSO, and CAR T cells were compared to untransduced T cells (UT), wherein the LNCAP and/or CAR T or UT cells were pre-treated (PreTx) with enzalutamide. Androgen receptor inhibition in LNCAP cells treated with either media, DMSO, or 5 uM enzalutamide was validated by qPCR of downstream target expression (FIG. 17C). Enzalutamide did not appear to have an effect on CAR T killing capacity or IFNγ release when co-cultured with LNCAP cells.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

All publications, patents, and patent applications disclosed herein are incorporated by reference to the same extent as if each individual publication, patent or patent application was specifically and individually indicated to be incorporated by reference.

1. A method for inhibiting the growth of a tumor cell comprising contacting said tumor cell with:
a) a T cell comprising (i) a polynucleotide encoding a chimeric antigen receptor (CAR) that binds an epitope on human six transmembrane epithelial antigen of prostate-2 (STEAP2); and
b) at least one androgen receptor antagonist, in an amount effective to inhibit tumor cell growth;
wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

2. A method for treating a cancer comprising tumor cells in a subject comprising administering to said subject in need thereof a therapeutically effective amount of:
a) a T cell comprising (i) a polynucleotide encoding a chimeric antigen receptor (CAR) that binds an epitope on human six transmembrane epithelial antigen of prostate-2 (STEAP2); and
b) at least one androgen receptor antagonist;
wherein the CAR comprises an antigen-binding domain comprising a VH and a VL, wherein the VH comprises a VH-CDR1, a VH-CDR2, a VH-CDR3, and wherein the VL comprises a VL-CDR1, a VL-CDR2, and VL-CDR3; and wherein the VL-CDR1 comprises the amino acid sequence set forth in SEQ ID NO: 1, the VL-CDR2 comprises the amino acid sequence set forth in SEQ ID NO: 2, the VL-CDR3 comprises the amino acid sequence set forth in SEQ ID NO: 3, the VH-CDR1 comprises the amino acid sequence set forth in SEQ ID NO: 4, the VH-CDR2 comprises the amino acid sequence set forth in SEQ ID NO: 5, and the VH-CDR3 comprises the amino acid sequence set forth in SEQ ID NO: 6.

3. (canceled)

4. The method of claim 1, wherein the VH comprises the amino acid sequence set forth in SEQ ID NO: 7, and the VL comprises the amino acid sequence set forth in SEQ ID NO: 8.

5. The method of claim 1, wherein the polynucleotide further encodes an armoring molecule and said armoring molecule comprises a dominant-negative TGF-β receptor type 2 (TGFβRIIDN).

6. (canceled)

7. The method of claim 1, wherein the armoring molecule comprises the amino acid sequence set forth in SEQ ID NO: 10.

8. (canceled)

9. The method of claim 1, wherein the polynucleotide encoding the CAR comprises the nucleotide sequence set forth in SEQ ID NO: 11.

10. The method of claim 1, wherein the polynucleotide encoding the CAR comprises the nucleotide sequence set forth in SEQ ID NO: 11, and the polynucleotide encoding the armoring molecule comprises the nucleotide sequence set forth in SEQ ID NO: 9.

11. The method of claim 1, wherein the polynucleotide encoding the CAR and the polynucleotide encoding the armoring molecule are operably linked under the control of a single promoter.

12. The method of claim 1, wherein the polynucleotide encoding the CAR and the polynucleotide encoding the armoring molecule are operably linked by an IRES.

13. The method of claim 1, wherein the polynucleotide encoding the CAR and the polynucleotide encoding the armoring molecule are linked by a nucleotide sequence encoding a cleavable peptide linker.

14. The method of claim 13, wherein the cleavable peptide linker is a self-cleaving peptide linker.

15. The method of claim 13, wherein the cleavable peptide linker comprises a T2A peptide.

16. The method of claim 13, wherein the cleavable peptide linker comprises SEQ ID NO: 13.

17. (canceled)

18. The method of claim 1, wherein the polynucleotide comprises the nucleotide sequence set forth in SEQ ID NO: 14.

19. The method of claim 1, wherein the T cell comprises an amino acid sequence that comprises the amino acid sequence set forth in SEQ ID NO: 12.

20. The method of claim 1, wherein the T cell comprises an amino acid sequence that comprises the amino acid sequence set forth in SEQ ID NO: 15.

21. The method of claim 1, wherein the androgen receptor antagonist is enzalutamide, apalutamide, darolutamide, abiraterone, bicalutamide, nilutamide, flutamide, proxalutamide, or a combination thereof.

22. (canceled)

23. (canceled)

24. The method of claim 2, wherein the androgen receptor antagonist is administered orally.

25. (canceled)

26. (canceled)

27. (canceled)

28. (canceled)

29. (canceled)

30. The method of claim 1, wherein the T cell is a CD8+ T cell.

31. The method of claim 1, wherein the tumor cells are prostate tumor cells, optionally wherein the prostate tumor cells are metastatic, recurrent, or relapsed.

32. (canceled)

33. (canceled)

34. (canceled)

35. (canceled)

36. (canceled)

37. (canceled)

38. (canceled)


SEQUENCE LISTING (22 sequences)

SEQ ID NO: 1 - protein, 11 aa, synthetic construct
  source 1..11: note = STEAP2 VL CDR1 (40A3)
RASQSVNSNLA

SEQ ID NO: 2 - protein, 7 aa, synthetic construct
  source 1..7: note = STEAP2 VL CDR2
GASTRAT

SEQ ID NO: 3 - protein, 9 aa, synthetic construct
  source 1..9: note = STEAP2 VL CDR3
QQYNNWPFT

SEQ ID NO: 4 - protein, 7 aa, synthetic construct
  source 1..7: note = STEAP2 VH CDR1
RNSAVWN

SEQ ID NO: 5 - protein, 18 aa, synthetic construct
  source 1..18: note = STEAP2 VH CDR2
RTYYRSKWYNDYAVSVKS

SEQ ID NO: 6 - protein, 13 aa, synthetic construct
  source 1..13: note = STEAP2 VH CDR3
GLLQNNFYYYMDV

SEQ ID NO: 7 - protein, 125 aa, synthetic construct
  source 1..125: note = STEAP2 VH
QVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 8 - protein, 107 aa, synthetic construct
  source 1..107: note = STEAP2 VL
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIK

SEQ ID NO: 9 - DNA, 582 bp, synthetic construct
  source 1..582: mol_type = other DNA; note = Dominant-negative TGF-beta receptor type 2 nucleic acid sequence
atgggacgcgggctgcttcgaggactctggccacttcatatcgtgttgtggactcgcatcgcttcaaccattccgccgcacgtgcagaagtccgtgaacaatgacatgatcgtgaccgacaacaacggtgcagtgaagttcccacagctgtgcaagttctgcgatgtcagattcagcacttgcgacaaccagaagtcctgcatgtcaaactgctccatcacctccatctgcgagaagcctcaagaggtctgcgtggccgtgtggcggaagaacgacgagaacatcaccctggaaaccgtgtgccacgatccgaagctgccttatcacgacttcattctggaagatgccgcctcgcccaagtgtatcatgaaagaaaagaaaaagcccggagaaacgttcttcatgtgctcgtgtagctccgacgagtgcaacgacaacattatctttagcgaagagtacaacacttccaaccctgacctcctgctcgtgatttttcaagtcaccggcatttccctgctgcccccgctgggcgtggcgatctcggtgatcattatcttctactgttaccgggtcaataggcag

SEQ ID NO: 10 - protein, 194 aa, synthetic construct
  source 1..194: note = Dominant-negative TGF-beta receptor type 2 amino acid sequence
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 11 - DNA, 1410 bp, synthetic construct
  source 1..1410: mol_type = other DNA; note = STEAP2 BZ CAR nucleic acid sequence
atgctgctccttgtcacatcactgctgctctgcgaactgccccaccctgcattcctcctgatccccgagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctccggctccgaatcaaaatacggtccgccatgcccaccgtgccccttctgggtgctcgtggtcgtcggaggggttctggcctgctactccctgctggtcaccgtggcgtttatcatcttctgggtgaagcggggaaggaagaagctactgtacattttcaagcagcctttcatgcggcctgtgcagaccacccaggaagaggacggctgttcctgccggttccccgaggaagaggaagggggttgcgagctgcgcgtgaagttcagcaggagcgctgatgccccagcgtaccaacaggggcaaaaccagttgtacaacgaactgaaccttggtcggcgcgaagagtacgacgtgcttgacaagcgccgcggcagagatcccgagatgggtggaaagccgcggcggaagaatccgcaggaagggctctacaacgagctccagaaggacaagatggccgaagcctacagcgaaatcgggatgaagggcgaaagacgccggggaaaaggacacgacggactgtaccaggggttgtcgaccgcgaccaaggacacctacgacgccctgcatatgcaagccttgccgccgagatga

SEQ ID NO: 12 - protein, 469 aa, synthetic construct
  source 1..469: note = STEAP2 BZ CAR amino acid sequence
MLLLVTSLLLCELPHPAFLLIPEIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 21 aa, synthetic construct
  source 1..21: note = T2A Peptide
GSGEGRGSLLTCGDVEENPGP

SEQ ID NO: 14 - DNA, 2052 bp, synthetic construct
  source 1..2052: mol_type = other DNA; note = Dominant-negative TGF-beta receptor type 2 nucleic acid sequence
atgctgctccttgtcacatcactgctgctctgcgaactgccccaccctgcattcctcctgatccccgagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctccggctccgaatcaaaatacggtccgccatgcccaccgtgccccttctgggtgctcgtggtcgtcggaggggttctggcctgctactccctgctggtcaccgtggcgtttatcatcttctgggtgaagcggggaaggaagaagctactgtacattttcaagcagcctttcatgcggcctgtgcagaccacccaggaagaggacggctgttcctgccggttccccgaggaagaggaagggggttgcgagctgcgcgtgaagttcagcaggagcgctgatgccccagcgtaccaacaggggcaaaaccagttgtacaacgaactgaaccttggtcggcgcgaagagtacgacgtgcttgacaagcgccgcggcagagatcccgagatgggtggaaagccgcggcggaagaatccgcaggaagggctctacaacgagctccagaaggacaagatggccgaagcctacagcgaaatcgggatgaagggcgaaagacgccggggaaaaggacacgacggactgtaccaggggttgtcgaccgcgaccaaggacacctacgacgccctgcatatgcaagccttgccgccgagaggatccggagaggggaggggaagcctcctcacttgcggcgatgtggaggaaaacccgggtcctatgggacgcgggctgcttcgaggactctggccacttcatatcgtgttgtggactcgcatcgcttcaaccattccgccgcacgtgcagaagtccgtgaacaatgacatgatcgtgaccgacaacaacggtgcagtgaagttcccacagctgtgcaagttctgcgatgtcagattcagcacttgcgacaaccagaagtcctgcatgtcaaactgctccatcacctccatctgcgagaagcctcaagaggtctgcgtggccgtgtggcggaagaacgacgagaacatcaccctggaaaccgtgtgccacgatccgaagctgccttatcacgacttcattctggaagatgccgcctcgcccaagtgtatcatgaaagaaaagaaaaagcccggagaaacgttcttcatgtgctcgtgtagctccgacgagtgcaacgacaacattatctttagcgaagagtacaacacttccaaccctgacctcctgctcgtgatttttcaagtcaccggcatttccctgctgcccccgctgggcgtggcgatctcggtgatcattatcttctactgttaccgggtcaataggcag

SEQ ID NO: 15 - protein, 684 aa, synthetic construct
  source 1..684: note = STEAP2 BZ CAR-T2A-Dominant-negative TGF-beta receptor type 2 amino acid sequence
MLLLVTSLLLCELPHPAFLLIPEIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSSGSESKYGPPCPPCPFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGEGRGSLLTCGDVEENPGPMGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQ

SEQ ID NO: 16 - protein, 252 aa, synthetic construct
  source 1..252: note = STEAP2 scFv amino acid sequence
EIVMTQSPATLSVSPGERATLSCRASQSVNSNLAWYQQKPGQAPRLLIYGASTRATGIPARFSGSGSGTEFTLTISSLQSEDFAVYYCQQYNNWPFTFGPGTKVDIKGGGGSGGGGSGGGGSGGGGSQVQLQQSGPGLVKPSQTLSLTCAISGDSVSRNSAVWNWIRQSPSRGLEWLGRTYYRSKWYNDYAVSVKSRITINPDTSKNQFSLQVNSVTPEDTAVYYCARGLLQNNFYYYMDVWGKGTTVTVSS

SEQ ID NO: 17 - protein, 42 aa, synthetic construct
  source 1..42: note = 4-1BB Activation Domain (B domain)
KRGRKKLLYIFKQPFMRPVQTTQEEDGCSCRFPEEEEGGCEL

SEQ ID NO: 18 - protein, 112 aa, synthetic construct
  source 1..112: note = CD3zeta Domain (z domain)
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 41 aa, synthetic construct
  source 1..41: note = CD28 Co-stimulatory Domain
RSKRSRLLHSDYMNMTPRRPGPTRKHYQPYAPPRDFAAYRS

SEQ ID NO: 20 - protein, 27 aa, synthetic construct
  source 1..27: note = CD28 Transmembrane Domain
FWVLVVVGGVLACYSLLVTVAFIIFWV

SEQ ID NO: 21 - protein, 12 aa, synthetic construct
  source 1..12: note = IgG4P Hinge (S228P)
ESKYGPPCPPCP

SEQ ID NO: 22 - DNA, 756 bp, synthetic construct
  source 1..756: mol_type = other DNA; note = STEAP2 scFv nucleic acid sequence
gagattgtgatgacccagagccctgcaactctgagcgtgtcacccggagaaagggccactctgtcgtgtcgagcatcgcagtccgtgaactccaatctcgcctggtaccagcagaagcctgggcaggccccgaggctgctcatctacggtgcctccacgagagccacgggaattccagcgcgctttagcggatccggctcgggaaccgagttcacccttaccatctcatcgctgcaatccgaagatttcgccgtgtattactgtcaacagtacaacaactggccgttcacctttggcccgggaactaaggtcgacatcaagggcggcgggggctctgggggtggcggaagcggcggcggcggatccggtggcggcggaagccaagtgcagctgcagcagtccggacccggactcgtgaagccgtcccagactctgtccctgacttgcgcgatttccggcgattccgtgtcccgcaactccgctgtgtggaactggatccggcagtcgccttcgagaggactggagtggctgggacggacctactaccgctcaaaatggtataacgactatgctgtgtccgtcaagagccgcatcaccattaaccccgatacctccaagaaccagttcagtctgcaagtcaacagcgtgactcctgaggacaccgccgtgtactactgcgcccggggtctgctgcaaaacaacttctactactacatggacgtctggggaaagggaactactgtgaccgtgtcctcc